(19) United States
(12) Patent Application Publication
(10) Pub. No.: US 2017/0281744 A1
(43) Pub. Date: Oct. 5, 2017

(54) METHOD OF TREATMENT

(71) Applicant: GlaxoSmithKline Biologicals SA, Rixensart (BE)

(72) Inventors: Ralph Leon BIEMANS, Rixensart (BE); Dominique BOUTRIAU, Rixensart (BE); Philippe DENOEL, Rixensart (BE); Pierre DUVIVIER, Rixensart (BE); Carine GORAJ, Rixensart (BE)

(21) Appl. No.: 15/529,094

(22) PCT Filed: Dec. 8, 2015
(86) PCT No.: PCT/EP2015/079023

§ 371 Date: May 24, 2017

(30) Foreign Application Priority Data
Dec. 10, 2014 (GB) 1421980.2
Dec. 10, 2014 (GB) 1421982.8

(51) Int. Cl. A61K 39/085; A61K 45/06; C07K 14/31

(52) U.S. Cl. CPC A61K 39/085; C07K 14/31; A61K 45/06; A61K 2039/6037

(57) ABSTRACT

The application discloses a method of immunising against Staphylococcus aureus infection comprising a step of administering to a human patient a single dose of an immunogenic composition comprising; (i) a Staphylococcus aureus ClfA protein or immunogenic fragment thereof at a dose of 5-50, 10-30, 5-15 or 20-40 μg and (ii) a pharmaceutically acceptable excipient; wherein the pH of the composition is pH 5.0-pH 8.0. The application further discloses an immunogenic composition comprising; (i) a S. aureus Type 5 capsular saccharide conjugated to a carrier protein, (ii) a S. aureus Type 8 capsular saccharide conjugated to a carrier protein, (iii) a ClfA protein or immunogenic fragment thereof, (iv) an alpha toxoid, and (v) a pharmaceutically acceptable excipient; wherein the pH of the immunogenic composition is pH 5.0-pH 8.0

### TECHNICAL FIELD

The present invention relates to the field of Staphylococcal immunogenic compositions and vaccines, their manufacture and the use of such compositions in medicine. More particularly, it relates to the use of a Clumping Factor A (ClfA) protein or fragment thereof (preferably an immunogenic fragment thereof) thereof, optionally from S. aureus. Such a ClfA protein or fragment thereof may be combined with further staphylococcal proteins and/or capsular saccharides from S. aureus antigens to form multivalent compositions. A further aspect of the invention relates to a use of alpha toxoid or fragment thereof from S. aureus. Alpha toxoid may be combined with further staphylococcal proteins and/or capsular saccharide from S. aureus to form multivalent compositions.

### BACKGROUND

Staphylococcus aureus (S. aureus) are commensal, Gram-positive bacteria which colonize the nares, axilla, pharynx and other mucosal and skin surfaces of about 30% of human subjects. S. aureus is estimated to be responsible for 20-25% of all healthcare associated infections (Wisplinghoff et al Clin Infect. Dis. 2004; 39; 309-317), resulting in three times the length of hospital stay and a 5-fold higher risk of in-hospital death for infected patients compared to patients without such infections (Noskin et al Arch. Intern. Med. 2005; 165; 1756-1761). S. aureus infections can be associated with in-hospital mortality rates of up to 25%. Historically, S. aureus has been associated mainly with nosocomial infections. The seriousness of such infections has increased with the recent dramatic increase in S. aureus infection associated with antibiotic resistance. Staphylococcus aureus is the most common cause of nosocomial infections with a significant morbidity and mortality (Romero-Vivas et al 1995, Infect. Dis. 21; 1417). It is the cause of some cases of osteomyelitis, endocarditis, septic arthritis, pneumonia, abscesses and toxic shock syndrome.

Passive immunotherapy involving administration of polyclonal antisera against staphylococcal antigens has been investigated (WO 00/15238, WO 00/12132) as well as immunotherapy using a monoclonal antibody against lipoteichoic acid (WO 98/57994). However as yet, none have been licensed for use. Several immunotherapy candidates failed to show efficacy in humans. These include; Altastaph (Nabi Biopharmaceuticals) containing CP5 and CP8 antibodies purified from subjects vaccinated with StaphVAX™ (investigational vaccine developed and trademarked by Nabi Biopharmaceuticals, Rockville, Md., USA; Veronate (Inhibitex), polyclonal antibodies targeting S. aureus clumping factor A (ClfA) and S. epidermidis adhesion SdrG; Aurexis (Tefibazumab, Inhibitex), monoclonal antibodies targetting ClfA; Aurograb (NeuTec Pharma), single chain antibodies against an ATP-binding cassette transporter; and Pagibaximab (Biosynexus), a monoclonal anti-lipoteichoic acid antibody (Dejonge et al J. Paediatrics 2007; 151; 260-265, Rupp et al Antimicrob. Agents Chemother. 2007; 51; 4249-4254).

Active vaccination to generate a polyclonal immune response against staphylococci has also been investigated. One approach reported in WO 03/61558 uses conjugates of S. aureus Type 5 and Type 8 capsular polysaccharides conjugated to Pseudomonas exoprotein A (StaphVAX—Nabi Biopharmaceuticals). A further approach used a S. aureus IsdB protein (V710—Merck & Co) but failed to demonstrate efficacy (Fowler et al 2013; JAMA 309; 1368-1378).

### DESCRIPTION OF FIGURES

FIG. 1—Percentage of subjects experiencing pain after 1 or 2 doses of the 4 component (4C) vaccine. In each formulation grouping, the first three columns provide the % of subjects experiencing pain after a single dose with the first column representing all reports of pain, the second column representing pain above or equal to grade 2 and the third column representing grade 3 pain. The 4^{th}, 5^{th} and 6^{th} columns show the same information after the second dose.

FIG. 2—Percentage of subjects experiencing redness after 1 or 2 doses of the 4C vaccine. In each formulation grouping, the first three columns provide the % of subjects experiencing redness after a single dose with the first column representing all reports of redness, the second column representing over 50 mm of redness and the third column representing over 100 mm of redness. The 4^{th}, 5^{th} and 6^{th} columns show the same information after the second dose.

FIG. 3—Percentage of subjects experiencing swelling after 1 or 2 doses of the 4C vaccine. In each formulation grouping, the first three columns provide the % of subjects experiencing swelling after a single dose with the first column representing all reports of swelling, the second column representing over 50 mm of swelling and the third column representing over 100 mm of swelling. The 4^{th}, 5^{th} and 6^{th} columns show the same information after the second dose.

FIG. 4—Immunogenicity results for antibodies raised against S. aureus Type 5 capsular polysaccharide. The geometric mean antibody concentration (GMC) results of a Luminex assay detecting antibodies against Type 5 capsular polysaccharide at various time points after the first and second immunisations are shown. The time points chosen are day 0 before immunisation, day 7 after one immunisation, day 14 after one immunisation, day 30 after one immunisation, day 7 after two immunisations (corresponding to day 37 on the graph), day 14 after two immunisations (corresponding to day 44 on the graph) and day 30 after two immunisations (corresponding to day 60 on the graph). For each time point, the results are presented in the order (left to right) of, 5/10, 5/10AS, 10/30, 10/30AS and saline.

FIG. 5—Immunogenicity results for antibodies raised against S. aureus Type 8 capsular polysaccharide. The GMC results of a Luminex assay detecting antibodies against Type 8 capsular polysaccharide at various time points after the first and second immunisations are shown. The time points chosen are day 0 before immunisation, day 7 after one immunisation, day 14 after one immunisation, day 30 after one immunisation, day 7 after two immunisations (corresponding to day 37 on the graph), day 14 after two immunisations (corresponding to day 44 on the graph) and day 30 after two immunisations (corresponding to day 60 on the graph). For each time point, the results are presented in the order (left to right) of, 5/10, 5/10AS, 10/30, 10/30AS and saline.

FIG. 6—Immunogenicity results for antibodies raised against S. aureus alpha toxoid. The GMC results of a Luminex assay detecting antibodies against alpha toxoid at various time points after the first and second immunisations are shown. The time points chosen are day 0 before immunisation, day 7 after one immunisation, day 14 after one immunisation, day 30 after one immunisation, day 7 after two immunisations (corresponding to day 37 on the graph), day 14 after two immunisations (corresponding to day 44 on the graph) and day 30 after two immunisations (corresponding to day 60 on the graph). For each time point, the results are presented in the order (left to right) of, 5/10, 5/10AS, 10/30, 10/30AS and saline.

FIG. 7—Immunogenicity results for antibodies raised against S. aureus ClfA. The GMC results of an ELISA detecting antibodies against ClfA at various time points after the first and second immunisations are shown. The time points chosen are day 0 before immunisation, day 7 after one immunisation, day 14 after one immunisation, day 30 after one immunisation, day 7 after two immunisations (corresponding to day 37 on the graph), day 14 after two immunisations (corresponding to day 44 on the graph) and day 30 after two immunisations (corresponding to day 60 on the graph). For each time point, the results are presented in the order (left to right) of, 5/10, 5/10AS, 10/30, 10/30AS and saline.

FIG. 8—Immunogenicity results for S. aureus Type 5 capsular polysaccharide (panel A), S. aureus Type 8 capsular saccharide (panel B), alpha toxoid (panel C) and ClfA (Panel D) over a longer time period of day 0 to day 540, after 1, 2 or 3 immunisations.

### DETAILED DESCRIPTION

There are many problems associated with the development of a vaccine against S. aureus infection. The failure of vaccines relying on a single component (capsular polysaccharide or the IsdB protein) suggests that a more complex vaccine containing multiple components may be required to induce protective immunity. However, combining different antigens in an immunogenic composition can lead to interference occurring in the composition (Skurnik et al (2010) J. Clin. Invest. 120; 3220-3233). The identification of components to combine in a multivalent composition is therefore not straight forward. There remains a need to develop an effective vaccine against staphylococcal infection, especially in view of increasing frequency of multidrug resistant strains.

In the case of immunising against nosocomial staphylococcal infection, immunisation may often take place a short time only before hospitalisation or surgery or placement of an indwelling catheter. It would therefore be advantageous to achieve high levels of immunity with a single immunisation. The use of lower doses of conjugate also has advantages of relative efficiency of vaccine production and associated economic benefits.

Accordingly there is provided a method of immunising against Staphylococcus aureus infection comprising a step of administering to a human patient a single dose of an immunogenic composition comprising; (i) a Staphylococcus aureus ClfA protein or immunogenic fragment thereof at a dose of 5-50, 10-30, 5-15 or 20-40 μg and (ii) a pharmaceutically acceptable excipient; wherein the pH of the composition is pH 5.0-pH 8.0.

In a second aspect of the invention, there is provided immunogenic composition comprising; (i) a Staphylococcus aureus ClfA protein or immunogenic fragment thereof at a dose of 5-50, 10-30, 5-15 or 20-40 μg, and (ii) a pharmaceutically acceptable excipient; wherein the pH of the immunogenic composition is pH 5.0-8.0, for use in treatment or prevention of Staphylococcus aureus infection in which a human patient is immunised with a single dose of the immunogenic composition.

In a further aspect of the invention, there is provided a method of immunising against Staphylococcus aureus infection comprising a step of administering to a human patient a single dose of an immunogenic composition comprising; (i) a Staphylococcus aureus alpha toxoid protein or immunogenic fragment thereof at a dose of 5-50, 10-30, 5-15 or 20-40 μg, and (ii) a pharmaceutically acceptable excipient, wherein the immunogenic composition has a pH of pH 5.0-pH 8.0.

In a further aspect of the invention, there is provided an immunogenic composition comprising; (i) a Staphylococcus aureus alpha toxoid protein or immunogenic fragment thereof at a dose of 5-50, 10-30, 5-15 or 20-40 μg, and (ii) a pharmaceutically acceptable excipient; wherein the pH of the immunogenic composition is pH 5.0-pH 8.0, for use in treatment or prevention of Staphylococcus aureus infection in which a human patient is immunised with a single dose of the immunogenic composition.

In a further aspect of the invention, there is provided an immunogenic composition comprising; (i) a S. aureus Type 5 capsular saccharide conjugated to a carrier protein, (ii) a S. aureus Type 8 capsular saccharide conjugated to a carrier protein, (iii) a ClfA protein or immunogenic fragment thereof, (iv) an alpha toxoid, and (v) a pharmaceutically acceptable excipient; wherein the pH of the immunogenic composition is pH 5.0-pH 8.0.

In a further aspect of the invention, there is provided a vaccine comprising a S. aureus Type 5 capsular saccharide conjugated to a carrier protein, a S. aureus Type 8 capsular saccharide conjugated to a carrier protein, a ClfA protein or immunogenic fragment thereof and an alpha toxoid and a pharmaceutically acceptable excipient wherein the pH of the immunogenic composition is pH 5.0-pH 8.0.

In a further aspect of the invention, there is provided a process for making the immunogenic composition or the vaccine of the invention comprising the steps of a) conjugating a S. aureus Type 5 capsular saccharide to a carrier protein to form a S. aureus Type 5 capsular saccharide conjugate, b) conjugating a S. aureus Type 8 capsular saccharide conjugated to a carrier protein to form a S. aureus Type 8 capsular saccharide conjugate, and c) combining the S. aureus Type 5 capsular saccharide conjugate, the S. aureus Type 8 capsular saccharide conjugate, a ClfA protein or immunogenic fragment thereof and an alpha toxoid to form the immunogenic composition; wherein the pH of the immunogenic composition is pH 5.0-pH 8.0.

The present invention discloses a method of immunising against Staphylococcus aureus infection comprising a step of administering to a human patient a single dose of an immunogenic composition comprising; (i) a Staphylococcus aureus ClfA protein or immunogenic fragment thereof at a dose of 5-50, 10-30, 5-15 or 20-40 μg and (ii) a pharmaceutically acceptable excipient; wherein the pH of the composition is pH 5.0-pH 8.0.

Clumping factor A (ClfA) has been identified as a S. aureus fibrinogen binding protein (U.S. Pat. No. 6,008,341) and has been identified as a potential carrier protein for polysaccharides which could be used to immunise against staphylococcal infection (WO 04/80490). ClfA is a surface located protein and is an important virulence factor due to its property of binding to fibrinogen and contributing to the adhesion of S. aureus. ClfA contains a fibrinogen binding region. This region, known as the A domain is located towards the N-terminus of ClfA and comprises three separately folded subdomains known as N1, N2 and N3. The A domain is followed by a serine-aspartate repeat region and a cell wall and membrane spanning region which contains the LPXTG motif for sortase-promoted anchoring to the cell wall. ClfA binds to the C-terminus of the γ-chain of fibrinogen, and is thereby able to induce clumping of bacteria in fibrinogen solution (McDevitt et al (1997) Eur. J. Biochem. 247; 416-424. Amino acid residues 221-559 of ClfA correspond to the N2-N3 region which retains fibrinogen binding. Fragments containing amino acids 221-559 of ClfA are suitable fragments for use in the invention. Amino acid residues 532 to 538 correspond to the latching peptide region of ClfA. Each subdomain comprises nine β-strands that form a novel IgG-type fold. The fibrinogen γ-chain peptide binding site in ClfA is located in a hydrophobic groove at the junction between N2 and N3.

Recently, amino acids P336 and Y338 of ClfA have been recognised as fibrinogen binding sites, mutation of which led to the loss of fibrinogen binding (Josefsson et al 2008, PLOS One volume 3, Issue 5, page 1-7). SEQ ID NO: 8-12, 17 and 18 contain point mutations at positions 336 and 338. The loss of fibrinogen binding in these variants led to an increased ability to protect against septic death in immunised mice, leading to the conclusion that the vaccine potential of recombinant ClfA is improved by removing its ability to bind fibrinogen (WO 09/95453). However, variants with point mutations at only one of Y256, P336, Y338 or K389 also lose their ability to bind fibrinogen (Deivanayagam et al EMBO J, 21; 6660-6672 (2002)). These single point mutations are expected to show similarly improved immunogenicity thus single mutations may also be used in the invention. In an embodiment, the immunogenic composition further comprises a ClfA protein or immunogenic fragment thereof. The ClfA protein or immunogenic fragment thereof is optionally recombinant, isolated or purified.

In an embodiment, the ClfA protein is at least 80%, 85%, 90%, 93%, 95%, 96%, 97%, 98%, 99% or 100% identical to the polypeptide sequence of SEQ ID NO:3, 4, 5, 6 or 7 or 8-12 along the entire length of thereof, optionally along the entire length of SEQ ID NO: 6.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GAP program in the GCG program package (Devereux, J., et al., Nucleic Acids Research 12(1): 387 (1984)), BLASTP, BLASTN (Altschul, S. F. et al., J. Molec. Biol. 215: 403-410 (1990), and FASTA (Pearson and Lipman Proc. Natl. Acad. Sci. USA 85; 2444-2448 (1988). The BLAST family of programs is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following:

### Algorithm: Needleman and Wunsch, J. Mol Biol. 48: 443-453 (1970)

Comparison matrix: BLOSSUM62 from Henikoff and Henikoff,
Proc. Natl. Acad. Sci. USA. 89:10915-10919 (1992)

### Gap Penalty: 8

### Gap Length Penalty: 2

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following:

### Algorithm: Needleman and Wunsch, J. Mol Biol. 48: 443-453 (1970)

Comparison matrix: matches=+10, mismatch=0

### Gap Penalty: 50

### Gap Length Penalty: 3

Available as: The “gap” program from Genetics Computer Group, Madison Wis. These are the default parameters for nucleic acid comparisons.

Where a protein is specifically mentioned herein, it encompasses the native or recombinant, full-length protein or optionally a mature protein in which any signal sequence has been removed. The protein may be isolated directly from the staphylococcal strain or produced by recombinant DNA techniques. Immunogenic fragments of the protein may be incorporated into the immunogenic composition of the invention. These are fragments comprising at least 10 amino acids, at least 20 amino acids, at least 30 amino acids, at least 40 amino acids, at least 50 amino acids or at least 100 amino acids, taken contiguously from the amino acid sequence of the protein. In addition, such immunogenic fragments are typically immunologically reactive with antibodies generated against the Staphylococcal proteins or with antibodies generated by infection of a mammalian host with Staphylococci or contain T cell epitopes. In an embodiment, immunogenic fragments also includes fragments that when administered at an effective dose, (either alone or as a hapten bound to a carrier), elicit a protective immune response against Staphylococcal infection, optionally it is protective against S. aureus and/or S. epidermidis infection. Such an immunogenic fragment may include, for example, the protein lacking an N-terminal leader sequence, and/or a transmembrane domain and/or a C-terminal anchor domain. For ClfA, preferred fragments lack the SD repeat domain towards the C-terminus of ClfA (for example by using a fragment in which amino acids 555-927, 556-927, 557-927, 558-927, 559-927 or 560-927 are deleted). For ClfA and alpha toxoid, preferred fragments have the signal peptide removed to form the mature protein, optionally with an initial methionine residue at the N-terminus to allow recombinant expression.

In an embodiment, immunogenic compositions of the invention may contain fusion proteins or immunogenic fragments of ClfA. The fusion protein optionally contains heterologous sequences such as a provider of T-cell epitopes or purification tags, for example: β-galactosidase, glutathione-S-transferase, green fluorescent proteins (GFP), epitope tags such as FLAG, myc tag, poly histidine, or viral surface proteins such as influenza virus haemagglutinin, or bacterial proteins such as tetanus toxoid, diphtheria toxoid, CRM197. The fusion protein may be present in the immunogenic composition of the invention as a free protein or it may be a carrier protein linked to a saccharide.

In an embodiment, the invention also provides an immunogenic fragment of the ClfA protein that is, a contiguous portion of the ClfA polypeptide which has the same or substantially the same immunogenic activity as the polypeptide comprising the polypeptide sequence of SEQ ID NO:3. That is to say, the fragment (if necessary when coupled to a carrier) is capable of raising an immune response which recognises ClfA polypeptide. Such an immunogenic fragment may include, for example, the ClfA polypeptide lacking an N-terminal leader sequence, and/or the SD repeat domain toward the C-terminus of ClfA. In a preferred aspect the immunogenic fragment of ClfA comprises substantially all of the fibrinogen binding domain and has at least 85% identity, preferably at least 90% identity, more preferably at least 95% identity, most preferably at least 97-99% identity or 100% identity, to the amino acid sequence of any one of SEQ ID NO:4-12 over the entire length of said sequence.

Fragments may be “free-standing,” or comprised within a larger polypeptide of which they form a part or region, most preferably as a single continuous region in a single larger polypeptide.

Further fragments of ClfA include an isolated polypeptide comprising an amino acid sequence having at least 15, 20, 30, 40, 50 or 100 contiguous amino acids from the amino acid sequence of SEQ ID NO:3.

In an embodiment, the ClfA protein is a immunogenic fragment of ClfA comprising the N1 domain, the N2 domain, the N3 domain, the N1 and N2 domains, the N2 and N3 domains or the N1 and N2 and N3 domains. Optionally, the ClfA immunogenic fragment comprises the N2 and N3 domains and has an amino acid sequence at least 80%, 85%, 90%, 95%, 97%, 98%, 99% or 100% identical to the sequence of SEQ ID NO: 6, 7, 11, 12, 15, 16, 17 or 18. Optionally, the ClfA immunogenic fragment comprises N1, N2 and N3 domains and has an amino acid sequence at least 80%, 85%, 90%, 95%, 97%, 98%, 99% or 100% identical to the sequence of SEQ ID NO:4, 5, 9 or 10.

In an embodiment, the ClfA protein or immunogenic fragment thereof contains an amino acid substitution, deletion or insertion which reduces or abolishes the ability of ClfA to bind to fibrinogen. In an embodiment, the ability of ClfA to bind to fibrinogen is reduced by at least 10, 20, 30, 40, 50, 60, 70, 80, 90, 95 or 99%. Such a mutation is typically in the fibrinogen binding region at the N-terminus of ClfA. The mutation is optionally an amino acid substitution at at least one, two, three or four of amino acids Ala254, Tyr256, Pro336, Tyr338, Ile387, Lys389, Tyr474, Glu526 or Val527. In an embodiment, ClfA amino acid Pro336 is mutated. In an embodiment ClfA amino acid Tyr338 is mutated. In an embodiment Tyr338 is mutated to Ala. In an embodiment, both Pro336 and Tyr338 are mutated, optionally to Alanine or Serine. In an embodiment, ClfA contains two mutations with Pro336 mutated to Ser and Tyr 338 mutated to Ala.

In an embodiment, the ClfA protein or immunogenic fragment is present in the immunogenic composition as an unconjugated protein. Alternatively, it is present conjugated to the S. aureus Type 5 capsular saccharide or to the S. aureus Type 8 capsular saccharide. In such cases, ClfA may act as a carrier protein and an antigen.

In an embodiment, the ClfA protein or immunogenic fragment thereof is present in the immunogenic composition at a dose of 5-50, 10-30, 5-15, 20-40 or 30-40 μg.

Alpha toxin is an important virulence determinant produced by most strains of S. aureus. It is a pore forming toxin with haemolytic activity. Antibodies against alpha toxin have been shown to neutralise the detrimental and lethal effects of alpha toxin in animal models (Adlam et al 1977 Infect. Immun. 17; 250). Human platelets, endothelial cells and mononuclear cells are susceptible to the effects of alpha toxin. In order for alpha toxin to be used in an immunogenic composition, it is typically detoxified by chemical treatment or mutation to produce alpha toxoid.

In an embodiment, the immunogenic composition comprises an alpha toxoid. Optionally the alpha toxoid has an amino acid sequence at least 90%, 95%, 96%, 97%, 98% or 99% identical to SEQ ID NO:1, 2, 13 or 14.

The high toxicity of alpha toxin requires that it should be detoxified before being used as an immunogen. This can be achieved by chemical treatment, for instance by treating with formaldehyde, glutaraldehyde of other cross-linking reagents or by chemically conjugating it to bacterial polysaccharides as described above.

A further way of removing toxicity is to introduce point mutations that remove toxicity while retaining the immunogenicity of the toxin. The introduction of a point mutation at amino acid 35 of alpha toxin where a histidine residue is replaced with a leucine residue results in the removal of toxicity whilst retaining immunogenicity (Menzies and Kernodle 1996; Infect. Immun. 64; 1839). Histidine 35 appears to be critical for the proper oligomerization required for pore formation and mutation of this residue leads to loss of toxicity. The modification of histidine 35 may be a substitution with Lys, Arg, Ala, Leu or Glu. Point mutation of alpha toxin at Asp24, Lys37, His48, Lys58, Asp100, Ile107, Glu111, Met113, Asp127, Asp128, Gly130, Gly134, His144, Lys147, Gln150, Asp152, Phe153, Lys154, Val169, Asn173, Arg200, Asn214, Leu219 or His259 can optionally be used to reduce toxicity.

When incorporated into immunogenic compositions of the invention, alpha toxoid is optionally detoxified by mutation of His 35, for example by replacing His 35 with Leu or Arg. In an alternative embodiment, alpha toxoid is detoxified by conjugation to other components of the immunogenic composition, for example to S. aureus Type 5 polysaccharide and/or S. aureus Type 8 polysaccharide. In an embodiment, the alpha toxoid is detoxified by both the introduction of a point mutation and by conjugation to S. aureus Type 5 polysaccharide and/or S. aureus Type 8 polysaccharide.

In an embodiment, the immunogenic composition comprises alpha toxoid which contains a point mutation which decreases toxicity of alpha toxin, for example at amino acid 35. The alpha toxoid optionally contains a point mutation at amino acid 35 where histidine is replaced with an arginine amino acid.

In an embodiment, the alpha toxoid is present in the immunogenic composition as an unconjugated protein. Alternatively, the alpha toxoid is conjugated to the S. aureus Type 5 capsular saccharide and/or to the S. aureus Type 8 capsular saccharide.

In an embodiment, the alpha toxoid is present in the immunogenic composition at a dose of 5-50, 10-30, 5-15, 20-40 or 30-40 μg. In an embodiment, the ClfA and alpha toxoid are present at the same dose in the immunogenic composition. In an embodiment the saccharide dose of Type 5 and 8 capsular saccharide conjugates is higher than the protein dose of ClfA and alpha toxoid.

In an embodiment, the immunogenic composition further comprises a S. aureus Type 5 capsular saccharide conjugated to a carrier protein to form a S. aureus Type 5 capsular saccharide conjugate, wherein the S. aureus Type 5 capsular saccharide conjugate is administered at a saccharide dose of 3-50 μg, 3-25 μg, 3-20 μg, 3-12 μg, 5-50 μg, 5-25 μg, 5-20 μg, 5-12 μg, 5-10 μg, 7-20 μg, 7-15 μg or 8-12 μg.

In an embodiment, the immunogenic composition further comprises a S. aureus Type 8 capsular saccharide conjugated to a carrier protein to form a S. aureus Type 8 capsular saccharide conjugate, wherein the S. aureus Type 8 capsular saccharide conjugate is administered at a saccharide dose of 3-50 μg, 3-25 μg, 3-20 μg, 3-12 μg, 5-50 μg, 5-25 μg, 5-20 μg, 5-12 μg, 5-10 μg, 7-20 μg, 7-15 μg or 8-12 μg.

In an embodiment, the same saccharide dose of S. aureus Type 5 capsular saccharide conjugate and S. aureus Type 8 capsular saccharide conjugate is present in the immunogenic composition; for example, a 4, 5, 6, 7, 8, 9 or 10 μg saccharide dose of both Type 5 and Type 8 conjugates.

Most strains of S. aureus that cause infection in man contain either Type 5 or Type 8 polysaccharides. Approximately 60% of human strains are Type 8 and approximately 30% are Type 5. Jones Carbohydrate Research 340, 1097-1106 (2005) used NMR spectroscopy to identify the structures of the capsular polysaccharides as:

### Type 5

### →4)-β-D-ManNAcA-(1→4)-α-L-FucNAc(3OAc)-(1→3)-β-D-FucNAc-(1→

### Type 8

### →3)-β-D-ManNAcA(4OAc)-(1→3)-α-L-FucNAc(1→3)-α-D-FucNAc(1→

Polysaccharides may be extracted from the appropriate strain of S. aureus using methods well known to the skilled man, for instance as described in U.S. Pat. No. 6,294,177, WO 11/41003, WO 11/51917 or Infection and Immunity (1990) 58(7); 2367. For example, ATCC 12902 is a Type 5 S. aureus strain and ATCC 12605 is a Type 8 S. aureus strain.

Polysaccharides are of native size or alternatively may be reduced in size, for instance by microfluidisation, ultrasonic irradiation or by chemical treatment such as exposure to pH 5.0-3.0. The invention also covers oligosaccharides derived from the Type 5 and 8 polysaccharides from S. aureus. In an embodiment the S. aureus Type 5 capsular saccharide has a molecular weight of over 25 kDa, 30 kDa, 40 kDa, 50 kDa, 60 kDa, 70 kDa, 80 kDa or 90 kDa or between 25-125 kDa, 90-125 kDa, 30-100 kDa, 35-75 KDa or 40-70 kDa. In an embodiment the S. aureus Type 8 capsular saccharide has a molecular weight of over 25 kDa, 30 kDa, 40 kDa, 50 kDa, 60 kDa, 70 kDa, 80 kDa or 90 kDa or between 25-125 kDa, 90-125 kDa, 30-100 kDa, 35-75 KDa or 40-70 kDa.

In an embodiment, the carrier protein to which the Type 5 and/or Type 8 capsular saccharide is conjugated is selected from the group consisting of tetanus toxoid, diphtheria toxoid, CRM197, alpha toxoid, ClfA, and Pseudomonas aeruginosa exoprotein A. In a preferred embodiment, the carrier protein is CRM197. In a further preferred embodiment, alpha toxoid is used as carrier protein for Type 5 capsular saccharide and ClfA is used as carrier protein for Type 8 capsular saccharide.

The Type 5 and/or 8 capsular polysaccharide or oligosaccharides included in the immunogenic composition of the invention are O-acetylated. In an embodiment, the degree of O-acetylation of Type 5 capsular polysaccharide or oligosaccharide is 50-100%. 60-100%, 70-100%, 80-100%, 90-100%, 50-90%, 60-90%, 70-90%, 70-80% or 80-90%. In an embodiment, the degree of O-acetylation of Type 8 capsular polysaccharide or oligosaccharide is 10-100%, 20-100%, 30-100%, 40-100%, 50-100%. 60-100%, 70-100%, 80-100%, 90-100%, 50-90%, 60-90%, 70-90%, 70-80% or 80-90%. In an embodiment, the degree of O-acetylation of Type 5 and Type 8 capsular polysaccharides or oligosaccharides is 10-100%, 20-100%, 30-100%, 40-100%, 50-100%. 60-100%, 70-100%, 80-100%, 90-100%, 50-90%, 60-90%, 70-90%, 70-80% or 80-90%. In an embodiment, the Type 5 and/or Type 8 capsular saccharides are 80-100% or 100% 0-acetylated.

The degree of O-acetylation of the polysaccharide or oligosaccharide can be determined by any method known in the art, for example, by proton NMR (Lemercinier and Jones 1996, Carbohydrate Research 296; 83-96, Jones and Lemercinier 2002, J Pharmaceutical and Biomedical analysis 30; 1233-1247, WO 05/033148 or WO 00/56357). A further commonly used method is that described by Hestrin (1949) J. Biol. Chem. 180; 249-261 (the Hestrin method is preferred).

O-acetyl groups can be removed by hydrolysis, for example by treatment with a base such as anhydrous hydrazine (Konadu et al 1994; Infect. Immun. 62; 5048-5054) or treatment with 0.1N NaOH for 1-8 hours. In order to maintain high levels of O-acetylation on Type 5 and/or 8 polysaccharide or oligosaccharide, treatments which would lead to hydrolysis of the O-acetyl groups are minimised. For example treatment at extremes of pH are minimised.

Amongst the problems associated with the use of polysaccharides in vaccination, is the fact that polysaccharides per se are poor immunogens. Strategies, which have been designed to overcome this lack of immunogenicity, include the linking of the polysaccharide to large protein carriers, which provide bystander T-cell help. In an embodiment, the polysaccharides utilised in the invention are linked to a protein carrier which provide bystander T-cell help. Examples of these carriers which may be used for coupling to polysaccharide or oligosaccharide immunogens include the Diphtheria and Tetanus toxoids (DT, DT Crm197 and TT), Keyhole Limpet Haemocyanin (KLH), Pseudomonas aeruginosa exoprotein A (rEPA) and the purified protein derivative of Tuberculin (PPD), protein D from Haemophilus influenzae, pneumolysin or fragments of any of the above. Fragments suitable for use include fragments encompassing T-helper epitopes. In particular protein D fragment will optionally contain the N-terminal 1/3 of the protein. Protein D is an IgD-binding protein from Haemophilus influenzae (EP 0 594 610 B1).

A new carrier protein that would be particularly advantageous to use in the context of a staphylococcal vaccine is staphylococcal alpha toxoid. The native form may be conjugated to a polysaccharide since the process of conjugation reduces toxicity. Optionally a genetically detoxified alpha toxin such as the His35Leu or His 35 Arg variants are used as carriers since residual toxicity is lower. Alternatively the alpha toxin is chemically detoxified by treatment with a cross-linking reagent, formaldehyde or glutaraldehyde. The process of conjugation is an alternative chemical treatment which detoxifies alpha toxin. A genetically detoxified alpha toxin is optionally chemically detoxified, optionally by treatment with a cross-linking reagent, formaldehyde or glutaraldehyde to further reduce toxicity.

The polysaccharides may be linked to the carrier protein(s) by any known method (for example, by Likhite, U.S. Pat. No. 4,372,945 by Armor et al., U.S. Pat. No. 4,474,757, Anderson et al WO 10/151544, Berti et al WO 11/138636, and Jennings et al., U.S. Pat. No. 4,356,170). Optionally, CDAP conjugation chemistry is carried out (see WO 95/08348, WO 07/113222).

In CDAP, the cyanylating reagent 1-cyano-dimethylaminopyridinium tetrafluoroborate (CDAP) is optionally used for the synthesis of polysaccharide-protein conjugates. The cyanilation reaction can be performed under relatively mild conditions, which avoids hydrolysis of the alkaline sensitive polysaccharides. This synthesis allows direct coupling to a carrier protein.

The polysaccharide may be solubilized in water or a saline solution. CDAP may be dissolved in acetonitrile and added immediately to the polysaccharide solution. The CDAP reacts with the hydroxyl groups of the polysaccharide to form a cyanate ester. After the activation step, the carrier protein is added. Amino groups of lysine react with the activated polysaccharide to form an isourea covalent link. After the coupling reaction, a large excess of glycine is then added to quench residual activated functional groups. The product is then passed through a gel permeation column to remove unreacted carrier protein and residual reagents.

In an embodiment, the S. aureus Type 5 capsular saccharide and/or the S. aureus Type 8 capsular saccharide is directly conjugated to the carrier protein. However, the invention also encompasses conjugates where the Type 5 and/or 8 capsular saccharides are conjugated through a linker, for example an ADH linker.

In an embodiment, the S. aureus Type 5 capsular saccharide and/or the S. aureus Type 8 capsular saccharide is conjugated using a cyanylating reagent, for example CDAP. Alternatively, other conjugation processes such as reductive amination or carbodiimide (for example EDAC) chemistry.

In an embodiment, the ratio of polysaccharide to protein in the S. aureus Type 5 capsular saccharide conjugate is between 1:5 and 5:1 (w:w), 1:1 and 1:5 (w/w), 1:2 and 1:5 (w/w), 1:3 and 1:5 (w/w) 1:2 and 2:1 (w/w) or 1:1 and 1:2 (w/w). In an embodiment, the ratio of polysaccharide to protein in the S. aureus Type 8 capsular saccharide conjugate is between 1:5 and 5:1 (w:w), 1:1 and 1:5 (w/w), 1:2 and 1:5 (w/w), 1:3 and 1:5 (w/w) 1:2 and 2:1 (w/w) or 1:1 and 1:2 (w/w).

In an embodiment, the immunogenic composition of the invention is mixed with a pharmaceutically acceptable excipient, and optionally with an adjuvant to form an immunogenic composition or vaccine.

In an embodiment, the pharmaceutically acceptable excipient is buffered at pH5.0-pH8.0, pH5.5-pH7.0, pH5.0-pH6.5, pH6.5-pH7.0, preferably at pH6.0-pH 7.0 or more preferably pH6.0-pH6.5. In an embodiment, the buffering is through a buffer with a pKa of 5.0-7.0. In an embodiment, the pharmaceutically acceptable excipient comprises a histidine buffer or succinate buffer, optionally present at a concentration of 5 mM-50 mM, or preferably 5 mM-15 mM.

In an embodiment, the pharmaceutically acceptable excipient comprises NaCl, optionally at a concentration of 50-150 mM NaCl, 50-100 mM or 140-160 mM.

The vaccines of the present invention may be adjuvanted, particularly when intended for use in an elderly, immunocompromised or chronically ill populations (such as diabetes, end stage renal disease or other populations at high risk of staphylococcal infection) but also for use in infant populations. Suitable adjuvants include an aluminium salt such as aluminium hydroxide gel or aluminium phosphate or alum, but may also be other metal salts such as those of calcium, magnesium, iron or zinc. Oil in water emulsions, for example comprising metabolisable oil (for example squalene), emulsifying agent (for example polyoxyethylene sorbitan monooleate) and optionally a tocol (for example alpha tocopherol) are also suitable (WO 09/95453).

It is preferred that the adjuvant be selected to be a preferential inducer of a TH1 type of response. Such high levels of Th1-type cytokines tend to favour the induction of cell mediated immune responses to a given antigen, whilst high levels of Th2-type cytokines tend to favour the induction of humoral immune responses to the antigen.

The distinction of Th1 and Th2-type immune response is not absolute. In reality an individual will support an immune response which is described as being predominantly Th1 or predominantly Th2. However, it is often convenient to consider the families of cytokines in terms of that described in murine CD4+ve T cell clones by Mosmann and Coffman (Mosmann, T. R. and Coffman, R. L. (1989) TH1 and TH2 cells: different patterns of lymphokine secretion lead to different functional properties. (Annual Review of Immunology, 7, p 145-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of Il-4, IL-5, IL-6, IL-10. Suitable adjuvant systems which promote a predominantly Th1 response include: Monophosphoryl lipid A or a derivative thereof (or detoxified lipid A in general—see for instance WO2005107798), particularly 3-de-O-acylated monophosphoryl lipid A (3D-MPL) (for its preparation see GB 2220211 A); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen [Thoelen et al. Vaccine (1998) 16:708-14; EP 689454-B1].

A further system involves the combination of a monophosphoryl lipid A and a saponin derivative, particularly the combination of QS21 and 3D-MPL as disclosed in WO 94/00153, or a less reactogenic composition where the QS21 is quenched with cholesterol as disclosed in WO 96/33739. A further adjuvant formulation involving QS21, 3D-MPL and tocopherol in an oil in water emulsion is described in WO 95/17210. In one embodiment the immunogenic composition additionally comprises a saponin, which may be QS21. The formulation may also comprise an oil in water emulsion and tocopherol (WO 95/17210). Unmethylated CpG containing oligonucleotides (WO 96/02555) and other immunomodulatory oligonucleotides (WO0226757 and WO03507822) are also preferential inducers of a TH1 response and are suitable for use in the present invention.

However, the inventors have found that in a clinical trial, the addition of an oil in water emulsion adjuvant did not produce an increase in immunogenicity. In view of the increased reactogenicity which can be associated with the use of adjuvant, an embodiment of the invention uses an unadjuvanted immunogenic composition, for example an immunogenic composition in which none of the staphylococcal components present is adsorbed to an adjuvant or an immunogenic composition in which the staphylococcal components are not mixed with an oil in water emulsion adjuvant. The staphylococcal components comprise 1, 2, 3 or 4 of a S. aureus Type 5 capsular saccharide conjugate, a S. aureus Type 8 capsular saccharide conjugate, a ClfA protein or immunogenic fragment thereof and an alpha toxoid.

A further aspect of the invention is a vaccine comprising the immunogenic composition described above and a pharmaceutically acceptable excipient. The vaccine preparations of the present invention may be used to protect or treat a human susceptible to S. aureus infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts.

Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877.

The vaccines of the present invention may be stored in solution or lyophilized. Optionally the solution is lyophilized in the presence of a sugar such as sucrose, trehalose or lactose. It is typical that they are lyophilized and extemporaneously reconstituted prior to use. Lyophilizing may result in a more stable composition (vaccine).

The invention also encompasses method of making the immunogenic compositions and vaccines of the invention. In an embodiment, the process of the invention, is a method to make a vaccine comprising the steps of a) conjugating a S. aureus Type 5 capsular saccharide to a carrier protein to form a S. aureus Type 5 capsular saccharide conjugate, b) conjugating a S. aureus Type 8 capsular saccharide conjugated to a carrier protein to form a S. aureus Type 8 capsular saccharide conjugate, and c) combining the S. aureus Type 5 capsular saccharide conjugate, the S. aureus Type 8 capsular saccharide conjugate, a ClfA protein or immunogenic fragment thereof and an alpha toxoid to form the immunogenic composition. In an embodiment, the process comprises a further step of adding a pharmaceutically acceptable excipient.

The invention also encompasses method of treatment or staphylococcal infection, particularly hospital acquired nosocomial infections.

This immunogenic composition or vaccine of the invention is particularly advantageous to use in cases of elective surgery, particularly when the subjects are immunised with a single dose. Such patients will know the date of surgery in advance and can advantageously be inoculated in advance. In an embodiment, the subject is immunised with a single dose of the immunogenic composition of the invention 5-60, 6-40, 7-30 or 7-15 days before admission to hospital. In an embodiment, the subject is immunised with a single dose of the immunogenic composition of the invention 5-60, 6-40, 7-30 or 7-15 days before a planned hospital procedure, for example a surgical procedure such as a cardio-thoracic surgical procedure. Typically adults over 16 awaiting elective surgery are treated with the immunogenic compositions and vaccines of the invention. Alternatively children aged 3-16 awaiting elective surgery are treated with the immunogenic compositions and vaccines of the invention.

It is also possible to inoculate health care workers with the vaccine of the invention.

The vaccine preparations of the present invention may be used to protect or treat a human susceptible to S. aureus infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts.

An embodiment of the invention is a method of preventing or treating staphylococcal infection or disease comprising the step of administering the immunogenic composition or vaccine of the invention to a patient in need thereof.

A further embodiment of the invention is a use of the immunogenic composition of the invention in the manufacture of a vaccine for treatment or prevention of staphylococcal infection or disease, optionally post-surgery staphylococcal infection.

Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877.

The invention is further described in the following paragraphs:
1. A method of immunising against Staphylococcus aureus infection comprising a step of administering to a human patient a single dose of an immunogenic composition comprising; (i) a Staphylococcus aureus ClfA protein or immunogenic fragment thereof at a dose of 5-50, 10-30, 5-15 or 20-40 μg and (ii) a pharmaceutically acceptable excipient; wherein the pH of the composition is pH 5.0-pH 8.0.
2. The method of paragraph 1 wherein the ClfA protein or immunogenic fragment thereof is at least 90% identical to the polypeptide sequence of any one of SEQ ID NO:3-12 or 15-18 along the full length thereof.
3. The method of paragraph 1 or 2 wherein the ClfA protein or immunogenic fragment thereof is a fragment of ClfA comprising a N2 domain.
4. The method of paragraphs 1-3 wherein the ClfA protein or immunogenic fragment thereof is a fragment of ClfA comprising a N3 domain.
5. The method of any one of paragraphs 1-4 wherein the ClfA protein or immunogenic fragment thereof is a fragment of ClfA comprising a N1 domain.
6. The method of any one of paragraphs 1-5 wherein the ClfA protein or immunogenic fragment thereof comprises the N2-N3 domains and has a polypeptide sequence at least 90% identical to the sequence of SEQ ID NO: 6, 7, 11, 12, 15, 16, 17 or 18.
7. The method of any one of paragraphs 1-6 wherein the ClfA protein or immunogenic fragment thereof contains an amino acid substitution which reduces the ability of ClfA to bind to fibrinogen.
8. The method of paragraph 7 wherein the ClfA protein or immunogenic fragment thereof contains an amino acid substitution at at least one of amino acids Ala254, Tyr256, Pro336, Tyr338, Ile387, Lys389, Tyr474, Glu526 or Val527.
9. The method of paragraph 7 wherein amino acid Pro336 is mutated to Ser and/or Tyr338 is mutated to Ala.
10. The method of any one of paragraphs 1-9 wherein the ClfA protein or immunogenic fragment thereof is present in the immunogenic composition as an unconjugated protein.
11. The method of any one of paragraphs 1-10 wherein the ClfA protein or immunogenic fragment thereof is conjugated to the S. aureus Type 5 capsular saccharide.
12. The method of any one of paragraphs 1-11 wherein the ClfA protein or immunogenic fragment thereof is conjugated to the S. aureus Type 8 capsular saccharide.
13. The method of any one of paragraphs 10-12 wherein the ClfA protein or immunogenic fragment thereof is present in the immunogenic composition at a dose of 20-40 μg.
14. The method of any preceding paragraph wherein the immunogenic composition comprises an alpha toxoid.
15. The method of paragraph 14 wherein the alpha toxoid has an amino acid sequence at least 90% identical to SEQ ID NO:1, 2, 13 or 14.
16. The method of paragraph 14 or 15 wherein the alpha toxoid contains a point mutation which decreases toxicity of the alpha toxoid.
17. The method of paragraph 16 wherein the alpha toxoid contains a point mutation at amino acid 35.
18. The method of paragraph 17, wherein the point mutation at amino acid 35 replaces a histidine with an arginine amino acid.
19. The method of any one of paragraphs 14-18 wherein the alpha toxoid is present in the immunogenic composition as an unconjugated protein.
20. The method of any one of paragraphs 14-19 wherein the alpha toxoid is conjugated to a S. aureus Type 5 capsular saccharide.
21. The method of any one of paragraphs 14-20 wherein the alpha toxoid is conjugated to a S. aureus Type 8 capsular saccharide.
22. The method of any one of paragraphs 14-21 wherein the alpha toxoid is present in the immunogenic composition at a dose of 5-50, 10-30, 5-15 or 20-40 μg.
23. The method of any one of paragraph 14-22 wherein the ClfA and alpha toxoid are present at the same dose in the immunogenic composition.
24. The method of any one of paragraphs 1-23 wherein the immunogenic composition comprises a S. aureus Type 5 capsular saccharide conjugated to a carrier protein to form a S. aureus Type 5 capsular saccharide conjugate, wherein the S. aureus Type 5 capsular saccharide conjugate is administered at a saccharide dose of 3-50 μg, 5-25 μg, 3-20 μg, 3-12 μg, 5-10 μg, 7-20 μg, 7-15 μg or 8-12 μg.
25. The method of paragraph 24 wherein the immunogenic composition comprises a S. aureus Type 8 capsular saccharide conjugated to a carrier protein to form a S. aureus Type 8 capsular saccharide conjugate, wherein the S. aureus Type 8 capsular saccharide conjugate is administered at a saccharide dose of 3-50 μg, 5-25 μg, 3-20 μg, 3-12 μg, 5-10 μg, 7-20 μg, 7-15 μg or 8-12 μg.
26. The method of paragraph 24 or 25 wherein the S. aureus Type 5 capsular saccharide has a molecular weight of over 25 kDa, 40 kDa or 50 kDa or between 25-300 kDa, 50-250 kDa, 70-150 kDa, 25-125 kDa, 90-125 kDa, 30-100 kDa, 35-75 KDa or 40-70 kDa.
27. The method of any one of paragraphs 24-26 wherein the S. aureus Type 8 capsular saccharide has a molecular weight of over 25 kDa, 40 kDa or 50 kDa or between 25-300 kDa, 50-250 kDa, 70-150 kDa, 25-125 kDa, 90-125 kDa, 30-100 kDa, 35-75 KDa or 40-70 kDa.
28. The method of any one of paragraphs 24-27 wherein the carrier protein to which the Type 5 capsular saccharide is conjugated is selected from the group consisting of tetanus toxoid, diphtheria toxoid, CRM197, alpha toxoid, ClfA, Pseudomonas aeruginosa exoprotein A.
29. The method of any one of paragraphs 24-28 wherein the carrier protein to which the Type 8 capsular saccharide is conjugated is selected from the group consisting of tetanus toxoid, diphtheria toxoid, CRM197, alpha toxoid, ClfA and Pseudomonas aeruginosa exoprotein A.
30. The method of paragraph 28 or 29 wherein the carrier protein is tetanus toxoid or CRM197.
31. The method of any one of paragraphs 24-30 wherein the S. aureus Type 5 capsular saccharide and/or the S. aureus Type 8 capsular saccharide is 50-100% or 75-100% O-acetylated.
32. The method of any one of paragraphs 24-31 wherein the S. aureus Type 5 capsular saccharide S. aureus and/or the Type 8 capsular saccharide is directly conjugated to the carrier protein.
33. The method of any one of paragraphs 24-32 wherein the S. aureus Type 5 capsular saccharide is conjugated using a cyanylating reagent.
34. The method of any one of paragraphs 24-33 wherein the S. aureus Type 8 capsular saccharide is conjugated using a cyanylating reagent.
35. The method of paragraph 33 or 34 wherein the cyanylating reagent is CDAP.
36. The method of any one of paragraphs 24-35 wherein the ratio of polysaccharide to protein in the S. aureus Type 5 capsular saccharide conjugate is between 1:5 and 5:1 (w:w) or between 1:2 and 2:1 (w/w), 1:2 to 1:5 (w/w) or 1:1 and 1:2 (w/w).
37. The method of any one of paragraphs 25-36 wherein the ratio of polysaccharide to protein in the S. aureus Type 8 capsular saccharide conjugate is between 1:5 and 5:1 (w:w) or between 1:2 and 2:1 (w/w), 1:2 to 1:5 (w/w) or 1:1 and 1:2 (w/w).
38. The method of any one of paragraphs 24-37 wherein the same saccharide dose of S. aureus Type 5 capsular saccharide and S. aureus Type 8 capsular saccharide is present in the immunogenic composition.
39. The method of any preceding paragraphs wherein the pH of the immunogenic composition is pH 6.0-pH 7.0.
40. The method of any preceding paragraph wherein the pharmaceutically acceptable excipient comprises a histidine or succinate buffer.
41. The method of any preceding paragraph wherein the pharmaceutically acceptable excipient comprises NaCl.
42. The method of paragraph 41 wherein the pharmaceutically acceptable excipient comprises 50-150 mM NaCl.
43. The method of any preceding paragraph wherein the immunogenic composition does not contain an oil in water emulsion.
44. The method of any preceding paragraph wherein the immunogenic composition is unadjuvanted.
45. The method of any one of paragraphs 1-44 wherein the single dose of the immunogenic composition is administered 5-60, 6-40, 7-30 or 7-15 days before a planned hospital procedure.
46. An immunogenic composition comprising; (i) a Staphylococcus aureus ClfA protein or immunogenic fragment thereof at a dose of 5-50, 10-30, 5-15 or 20-40 μg, and (ii) a pharmaceutically acceptable excipient; wherein the pH of the immunogenic composition is pH 5.0-8.0, for use in treatment or prevention of Staphylococcus aureus infection in which a human patient is immunised with a single dose of the immunogenic composition.
47. The immunogenic composition for use in treatment or prevention of S. aureus infection of paragraph 46 wherein the ClfA protein or immunogenic fragment thereof is at least 90% identical to the polypeptide sequence of any one of SEQ ID NO:3-12 or 15-18 along the full length thereof.
48. The immunogenic composition for use in treatment or prevention of S. aureus infection of paragraph 46 or 47 wherein the ClfA protein or immunogenic fragment thereof is a fragment of ClfA comprising a N2 domain.
49. The immunogenic composition for use in treatment or prevention of S. aureus infection of paragraphs 46-48 wherein the ClfA protein or immunogenic fragment thereof is a fragment of ClfA comprising a N3 domain.
50. The immunogenic composition for use in treatment or prevention of S. aureus infection of any one of paragraphs 46-49 wherein the ClfA protein or immunogenic fragment thereof is a fragment of ClfA comprising a N1 domain.
51. The immunogenic composition for use in treatment or prevention of S. aureus infection of any one of paragraphs 46-50 wherein the ClfA protein or immunogenic fragment thereof comprises the N2-N3 domains and has a polypeptide sequence at least 90% identical to the sequence of SEQ ID NO: 6, 7, 11, 12, 15, 16, 17 or 18.
52. The immunogenic composition for use in treatment or prevention of S. aureus infection of any one of paragraphs 46-51 wherein the ClfA protein or immunogenic fragment thereof contains an amino acid substitution which reduces the ability of ClfA to bind to fibrinogen.
53. The immunogenic composition for use in treatment or prevention of S. aureus infection of paragraph 52 wherein the ClfA protein or immunogenic fragment thereof contains an amino acid substitution at at least one of amino acids Ala254, Tyr256, Pro336, Tyr338, Ile387, Lys389, Tyr474, Glu526 or Val527.
54. The immunogenic composition for use in treatment or prevention of S. aureus infection of paragraph 52 wherein amino acid Pro336 is mutated to Ser and/or Y338 is mutated to Ala.
55. The immunogenic composition for use in treatment or prevention of S. aureus infection of any one of paragraphs 46-54 wherein the ClfA protein or immunogenic fragment thereof is present in the immunogenic composition as an unconjugated protein.
56. The immunogenic composition for use in treatment or prevention of S. aureus infection of any one of paragraphs 46-55 wherein the ClfA protein or immunogenic fragment thereof is conjugated to the S. aureus Type 5 capsular saccharide.
57. The immunogenic composition for use in treatment or prevention of S. aureus infection of any one of paragraphs 46-56 wherein the ClfA protein or immunogenic fragment thereof is conjugated to the S. aureus Type 8 capsular saccharide.
58. The immunogenic composition for use in treatment or prevention of S. aureus infection of any one of paragraphs 46-57 wherein the ClfA protein or immunogenic fragment thereof is present in the immunogenic composition at a dose of 20-40 μg.
59. The immunogenic composition for use in treatment or prevention of S. aureus infection of any preceding paragraph wherein the immunogenic composition comprises an alpha toxoid.
60. The immunogenic composition for use in treatment or prevention of S. aureus infection of paragraph 59 wherein the alpha toxoid has an amino acid sequence at least 90% identical to SEQ ID NO:1, 2, 13 or 14.
61. The immunogenic composition for use in treatment or prevention of S. aureus infection of paragraph 59 or 60 wherein the alpha toxoid contains a point mutation which decreases toxicity of the alpha toxoid.
62. The immunogenic composition for use in treatment or prevention of S. aureus infection of paragraph 61 wherein the alpha toxoid contains a point mutation at amino acid 35.
63. The immunogenic composition for use in treatment or prevention of S. aureus infection of paragraph 62, wherein the point mutation at amino acid 35 replaces a histidine with an arginine amino acid.
64. The immunogenic composition for use in treatment or prevention of S. aureus infection of any one of paragraphs 59-63 wherein the alpha toxoid is present in the immunogenic composition as an unconjugated protein.
65. The immunogenic composition for use in treatment or prevention of S. aureus infection of any one of paragraphs 59-64 wherein the alpha toxoid is conjugated to the S. aureus Type 5 capsular saccharide.
66. The immunogenic composition for use in treatment or prevention of S. aureus infection of any one of paragraphs 59-65 wherein the alpha toxoid is conjugated to the S. aureus Type 8 capsular saccharide.
67. The immunogenic composition for use in treatment or prevention of S. aureus infection of any one of paragraphs 59-66 wherein the alpha toxoid is present in the immunogenic composition at a dose of 5-50, 10-30, 5-15 or 20-40 μg.
68. The immunogenic composition for use in treatment or prevention of S. aureus infection of any one of paragraph 59-67 wherein the ClfA and alpha toxoid are present at the same dose in the immunogenic composition.
69. The immunogenic composition for use in treatment or prevention of S. aureus infection of any one of paragraphs 46-68 wherein the immunogenic composition comprises a S aureus Type 5 capsular saccharide conjugated to a carrier protein to form a S. aureus Type 5 capsular saccharide conjugate, wherein the S. aureus Type 5 capsular saccharide conjugate is administered at a saccharide dose of 3-50 μg, 5-25 μg, 3-20 μg, 3-12 μg, 5-10 μg, 7-20 μg, 7-15 μg or 8-12 μg.
70. The immunogenic composition for use in treatment or prevention of S. aureus infection of paragraph 69 wherein the immunogenic composition comprises a S. aureus Type 8 capsular saccharide conjugated to a carrier protein to form a S. aureus Type 8 capsular saccharide conjugate, wherein the S. aureus Type 8 capsular saccharide conjugate is administered at a saccharide dose of 3-50 μg, 5-25 μg, 3-20 μg, 3-12 μg, 5-10 μg, 7-20 μg, 7-15 μg or 8-12 μg.
71. The immunogenic composition for use in treatment or prevention of S. aureus infection of paragraph 69 or 70 wherein the S. aureus Type 5 capsular saccharide has a molecular weight of over 25 kDa, 40 kDa or 50 kDa or between 25-300 kDa, 50-250 kDa, 70-150 kDa, 25-125 kDa, 90-125 kDa, 30-100 kDa, 35-75 KDa or 40-70 kDa.
72. The immunogenic composition for use in treatment or prevention of S. aureus infection of any one of paragraphs 69-71 wherein the S. aureus Type 8 capsular saccharide has a molecular weight of over 25 kDa, 40 kDa or 50 kDa or between 25-300 kDa, 50-250 kDa, 70-150 kDa, 25-125 kDa, 90-125 kDa, 30-100 kDa, 35-75 KDa or 40-70 kDa.
73. The immunogenic composition for use in treatment or prevention of S. aureus infection of any one of paragraphs 69-72 wherein the carrier protein to which the Type 5 capsular saccharide is conjugated is selected from the group consisting of tetanus toxoid, diphtheria toxoid, CRM197, alpha toxoid, ClfA, Pseudomonas aeruginosa exoprotein A.
74. The immunogenic composition for use in treatment or prevention of S. aureus infection of any one of paragraphs 69-73 wherein the carrier protein to which the Type 8 capsular saccharide is conjugated is selected from the group consisting of tetanus toxoid, diphtheria toxoid, CRM197, alpha toxoid, ClfA and Pseudomonas aeruginosa exoprotein A.
75. The immunogenic composition for use in treatment or prevention of S. aureus infection of paragraph 73 or 74 wherein the carrier protein is tetanus toxoid or CRM197.
76. The immunogenic composition for use in treatment or prevention of S. aureus infection of any one of paragraphs 69-75 wherein the S. aureus Type 5 capsular saccharide and/or the S. aureus Type 8 capsular saccharide is 50-100% or 75-100% 0-acetylated.
77. The immunogenic composition for use in treatment or prevention of S. aureus infection of any one of paragraphs 69-76 wherein the S. aureus Type 5 capsular saccharide S. aureus and/or the Type 8 capsular saccharide is directly conjugated to the carrier protein.
78. The immunogenic composition for use in treatment or prevention of S. aureus infection of any one of paragraphs 69-77 wherein the S. aureus Type 5 capsular saccharide is conjugated using a cyanylating reagent.
79. The immunogenic composition for use in treatment or prevention of S. aureus infection of any one of paragraphs 69-78 wherein the S. aureus Type 8 capsular saccharide is conjugated using a cyanylating reagent.
80. The immunogenic composition for use in treatment or prevention of S. aureus infection of paragraph 78 or 79 wherein the cyanylating reagent is CDAP.
81. The immunogenic composition for use in treatment or prevention of S. aureus infection of any one of paragraphs 69-80 wherein the ratio of polysaccharide to protein in the S. aureus Type 5 capsular saccharide conjugate is between 1:5 and 5:1 (w:w) or between 1:2 and 2:1 (w/w), 1:2 to 1:5 (w/w) or 1:1 and 1:2 (w/w).
82. The immunogenic composition for use in treatment or prevention of S. aureus infection of any one of paragraphs 69-81 wherein the ratio of polysaccharide to protein in the S. aureus Type 8 capsular saccharide conjugate is between 1:5 and 5:1 (w:w) or between 1:2 and 2:1 (w/w), 1:2 to 1:5 (w/w) or 1:1 and 1:2 (w/w).
83. The immunogenic composition for use in treatment or prevention of S. aureus infection of any one of paragraphs 69-82 wherein the same saccharide dose of S. aureus Type 5 capsular saccharide and S. aureus Type 8 capsular saccharide is present in the immunogenic composition.
84. The immunogenic composition for use in treatment or prevention of S. aureus infection of any one of paragraphs 46-83 wherein the pH of the immunogenic composition is pH 6.0-pH 7.0.
85. The immunogenic composition for use in treatment or prevention of S. aureus infection of any one of paragraphs 46-84 wherein the pharmaceutically acceptable excipient comprises a histidine or succinate buffer.
86. The immunogenic composition for use in treatment or prevention of S. aureus infection of any one of paragraphs 46-85 wherein the pharmaceutically acceptable excipient comprises NaCl.
87. The immunogenic composition for use in treatment or prevention of S. aureus infection of paragraph 86 wherein the pharmaceutically acceptable excipient comprises 50-150 mM NaCl.
88. The immunogenic composition for use in treatment or prevention of S. aureus infection of any one of paragraphs 46-87 wherein the immunogenic composition does not contain an oil in water emulsion.
89. The immunogenic composition for use in treatment or prevention of S. aureus infection of any one of paragraphs 46-88 wherein the immunogenic composition is unadjuvanted.
90. The immunogenic composition for use in treatment or prevention of S. aureus infection of any one of paragraphs 46-89 wherein the single dose of the immunogenic composition is administered 5-60, 6-40, 7-30 or 7-15 days before a planned hospital procedure.
91. A method of immunising against Staphylococcus aureus infection comprising a step of administering to a human patient a single dose of an immunogenic composition comprising; (i) a Staphylococcus aureus alpha toxoid protein or immunogenic fragment thereof at a dose of 5-50, 10-30, 5-15 or 20-40 μg, and (ii) a pharmaceutically acceptable excipient; wherein the immunogenic composition has a pH of pH 5.0-pH 8.0.
92. The method of paragraph 91, comprising a step of administering to a human patient a single dose of the immunogenic composition as recited in any one of paragraphs 1-44.
93. An immunogenic composition comprising; (i) a Staphylococcus aureus alpha toxoid protein or immunogenic fragment thereof at a dose of 5-50, 10-30, 5-15 or 20-40 μg, and (ii) a pharmaceutically acceptable excipient; wherein the pH of the immunogenic composition is pH 5.0-pH 8.0, for use in treatment or prevention of Staphylococcus aureus infection in which a human patient is immunised with a single dose of the immunogenic composition.
94. The immunogenic composition for use of paragraph 93, comprising the immunogenic composition for use of any one of paragraphs 47-90.
95. An immunogenic composition comprising; (i) a S. aureus Type 5 capsular saccharide conjugated to a carrier protein, (ii) a S. aureus Type 8 capsular saccharide conjugated to a carrier protein, (iii) a ClfA protein or immunogenic fragment thereof, (iv) an alpha toxoid, and (v) a pharmaceutically acceptable excipient; wherein the pH of the immunogenic composition is pH 5.0-pH 8.0.
96. The immunogenic composition of paragraph 95 wherein the S. aureus Type 5 capsular saccharide conjugate is present at a saccharide dose of 3-50 μg, 5-25 μg, 3-20 μg, 3-12 μg, 5-10 μg, 7-20 μg, 7-15 μg or 8-12 μg.
97. The immunogenic composition of paragraph 95 or 96 wherein the S. aureus Type 8 capsular saccharide conjugate is present at a saccharide dose of 3-50 μg, 5-25 μg, 3-20 μg, 3-12 μg, 5-10 μg, 7-20 μg, 7-15 μg or 8-12 μg.
98. The immunogenic composition of any one of paragraphs 95-97 wherein the S. aureus Type 5 capsular saccharide has a molecular weight of over 25 kDa, 40 kDa or 50 kDa or between 25-300 kDa, 50-250 kDa, 70-150 kDa, 25-125 kDa, 90-125 kDa, 30-100 kDa, 35-75 KDa or 40-70 kDa.
99. The immunogenic composition of any one of paragraphs 95-98 wherein the S. aureus Type 8 capsular saccharide has a molecular weight of over 25 kDa, 40 kDa or 50 kDa or between 25-300 kDa, 50-250 kDa, 70-150 kDa, 25-125 kDa, 90-125 kDa, 30-100 kDa, 35-75 KDa or 40-70 kDa.
100. The immunogenic composition of any one of paragraphs 95-99 wherein the carrier protein to which the Type 5 capsular saccharide is conjugated is selected from the group consisting of tetanus toxoid, diphtheria toxoid, CRM197, alpha toxoid, ClfA, Pseudomonas aeruginosa exoprotein A.
101. The immunogenic composition of any one of paragraphs 95-100 wherein the carrier protein to which the Type 8 capsular saccharide is conjugated is selected from the group consisting of tetanus toxoid, diphtheria toxoid, CRM197, alpha toxoid, ClfA and Pseudomonas aeruginosa exoprotein A.
102. The immunogenic composition of paragraph 100 or 101 wherein the carrier protein is tetanus toxoid or CRM197.
103. The immunogenic composition of any one of paragraphs 95-102 wherein the S. aureus Type 5 capsular saccharide and/or the S. aureus Type 8 capsular saccharide is 50-100% or 75-100% O-acetylated.
104. The immunogenic composition of any one of paragraphs 95-103 wherein the S. aureus Type 5 capsular saccharide and/or the S. aureus Type 8 capsular saccharide is directly conjugated to the carrier protein.
105. The immunogenic composition of any one of paragraphs 95-104 wherein the S. aureus Type 5 capsular saccharide is conjugated using a cyanylating reagent.
106. The immunogenic composition of any one of paragraphs 95-105 wherein the S. aureus Type 8 capsular saccharide is conjugated using a cyanylating reagent.
107. The immunogenic composition of paragraph 105 or 106 wherein the cyanylating reagent is CDAP.
108. The immunogenic composition of any one of paragraphs 95-107 wherein the ratio of polysaccharide to protein in the S. aureus Type 5 capsular saccharide conjugate is between 1:5 and 5:1 (w:w) or between 1:2 and 2:1 (w/w), 1:2 to 1:5 (w/w) or 1:1 and 1:2 (w/w).
109. The immunogenic composition of any one of paragraphs 95-108 wherein the ratio of polysaccharide to protein in the S. aureus Type 8 capsular saccharide conjugate is between 1:5 and 5:1 (w:w) or between 1:2 and 2:1 (w/w), 1:2 to 1:5 (w/w) or 1:1 and 1:2 (w/w).
110. The immunogenic composition of any one of paragraphs 95-109 wherein the same saccharide dose of S. aureus Type 5 capsular saccharide and S. aureus Type 8 capsular saccharide is present in the immunogenic composition.
111. The immunogenic composition of any one of paragraphs 95-110 wherein the immunogenic composition further comprises a ClfA protein or immunogenic fragment thereof.
112. The immunogenic composition of paragraph 111 wherein the ClfA protein or immunogenic fragment thereof is at least 90% identical to the amino acid sequence of any one of SEQ ID NO:3-12 or 15-18 along the full length of thereof.
113. The immunogenic composition of paragraph 111 or 112 wherein the ClfA protein or immunogenic fragment thereof is a fragment of ClfA comprising a N2 domain.
114. The immunogenic composition of paragraphs 111-113 wherein the ClfA protein or immunogenic fragment thereof is a fragment of ClfA comprising a N3 domain.
115. The immunogenic composition of any one of paragraphs 111-114 wherein the ClfA protein or immunogenic fragment thereof is a fragment of ClfA comprising a N1 domain.
116. The immunogenic composition of any one of paragraphs 111-115 wherein the ClfA protein or immunogenic fragment thereof comprises the N2-N3 domain and has an amino acid sequence at least 90% identical to the sequence of SEQ ID NO: 6, 7, 11, 12, 15, 16, 17 or 18.
117. The immunogenic composition of any one of paragraphs 111-116 wherein the ClfA protein or immunogenic fragment thereof contains an amino acid substitution which reduces the ability of ClfA to bind to fibrinogen.
118. The immunogenic composition of paragraph 117 wherein the ClfA protein or immunogenic fragment thereof contains an amino acid substitution at at least one of amino acids Ala254, Tyr256, Pro336, Tyr338, Ile387, Lys389, Tyr474, Glu526 or Val527.
119. The immunogenic composition of paragraph 118 wherein amino acid Pro336 is mutated to Ser and/or Tyr338 is mutated to Ala.
120. The immunogenic composition of any one of paragraphs 111-119 wherein the ClfA protein or immunogenic fragment is present in the immunogenic composition as an unconjugated protein.
121. The immunogenic composition of any one of paragraphs 111-120 wherein the ClfA protein or immunogenic fragment thereof is conjugated to the S. aureus Type 5 capsular saccharide.
122. The immunogenic composition of any one of paragraphs 111-121 wherein the ClfA protein or immunogenic fragment thereof is conjugated to the S. aureus Type 8 capsular saccharide.
123. The immunogenic composition of any one of paragraphs 111-122 wherein the ClfA protein or immunogenic fragment thereof is present in the immunogenic composition at a dose of 5-50, 10-30, 5-15 or 20-40 μg.
124. The immunogenic composition of paragraph 123 wherein the immunogenic composition has a volume of 0.5 ml.
125. The immunogenic composition of any one of paragraphs 95-124 wherein the immunogenic composition comprises an alpha toxoid.
126. The immunogenic composition of paragraph 125 wherein the alpha toxoid has an amino acid sequence at least 90% identical to SEQ ID NO:1, 2, 3 or 4.
127. The immunogenic composition of paragraph 125 or 126 wherein the alpha toxoid contains a point mutation which decreases toxicity of the alpha toxoid.
128. The immunogenic composition of paragraph 127 wherein the alpha toxoid contains a point mutation at amino acid 35.
129. The immunogenic composition of paragraph 128, wherein the point mutation at amino acid 35 replaces a histidine with an arginine or a leucine amino acid.
130. The immunogenic composition of any one of paragraphs 125-129 wherein the alpha toxoid is present in the immunogenic composition as an unconjugated protein.
131. The immunogenic composition of any one of paragraphs 125-130 wherein the alpha toxoid is conjugated to the S. aureus Type 5 capsular saccharide.
132. The immunogenic composition of any one of paragraphs 125-131 wherein the alpha toxoid is conjugated to the S. aureus Type 8 capsular saccharide.
133. The immunogenic composition of any one of paragraphs 125-132 wherein the alpha toxoid is present in the immunogenic composition at a dose of 5-50, 10-30, 5-15 or 20-40 μg.
134. The immunogenic composition of any one of paragraphs 125-133 wherein the ClfA and alpha toxoid are present at the same dose in the immunogenic composition.
135. The immunogenic composition of any one of paragraphs 95-134 wherein the immunogenic composition has a pH of pH 6.0-pH 7.0.
136. The immunogenic composition of any one of paragraphs 95-135 wherein the pharmaceutically acceptable excipient comprises a histidine or succinate buffer.
137. The immunogenic composition of any one of paragraphs 95-136 wherein the pharmaceutically acceptable excipient comprises NaCl.
138. The immunogenic composition of paragraph 137 wherein the pharmaceutically acceptable excipient comprises 50-150 mM NaCl.
139. The immunogenic composition of any one of paragraphs 95-138 wherein the immunogenic composition does not contain oil in water emulsion.
140. The immunogenic composition of any one of paragraphs 95-139 wherein the immunogenic composition is unadjuvanted.
141. A vaccine comprising the immunogenic composition of any one of paragraphs 95-140.
142. A process for making the immunogenic composition of any one of paragraphs 95-140 or the vaccine of paragraph 141 comprising the steps of a) conjugating a S. aureus Type 5 capsular saccharide to a carrier protein to form a S. aureus Type 5 capsular saccharide conjugate, b) conjugating a S. aureus Type 8 capsular saccharide conjugated to a carrier protein to form a S. aureus Type 8 capsular saccharide conjugate, and c) combining the S. aureus Type 5 capsular saccharide conjugate, the S. aureus Type 8 capsular saccharide conjugate, a ClfA protein or immunogenic fragment thereof and an alpha toxoid to form the immunogenic composition.
143. The immunogenic composition of any one of paragraphs 95-140 or the vaccine of paragraph 141 for use in the treatment or prevention of S. aureus infection in a human subject.
144. The immunogenic composition for use of paragraph 143 wherein the human subject is undergoing a surgical procedure, optionally cardio-thoracic surgery.
145. The immunogenic composition for use of paragraph 144 wherein the human subject is immunised with a single dose of the immunogenic compositions of any one of paragraphs 91-136 or the vaccine of paragraph 141 at a time point 5-60, 6-40, 7-30 or 7-15 days before undergoing the surgical procedure.
146. A method of treatment comprising the step of administering to a human subject at risk of developing a S. aureus infection an immunologically effective amount of the immunogenic composition of any one of paragraphs 95-140 or the vaccine of paragraph 141.
147. The method of paragraph 146 wherein the human subject is undergoing a surgical procedure, optionally cardio-thoracic surgery.
148. The method of paragraph 147 wherein the human subject is immunised with a single dose of the immunogenic compositions of any one of paragraphs 91-136 or the vaccine of paragraph 137 at a time point 5-60, 6-40, 7-30 or 7-15 days before undergoing the surgical procedure.

The terms “comprising”, “comprise” and “comprises” herein are intended by the inventors to be optionally substitutable with the terms “consisting of”, “consist of” and “consists of”, respectively, in every instance.

All references or patent applications cited within this patent specification are incorporated by reference herein.

In order that this invention may be better understood, the following examples are set forth. These examples are for purposes of illustration only, and are not to be construed as limiting the scope of the invention in any manner.

### EXAMPLES

### Example 1 Sequences of Proteins

SEQ ID NO: 1
MKTRIVSSVTTTLLLGSILMNPVANAADSDINIKTGTTDIGSNTTVKTGD
LVTYDKENGMHKKVFYSFIDDKNHNKKLLVIRTKGTIAGQYRVYSEEGAN
KSGLAWPSAFKVQLQLPDNEVAQISDYYPRNSIDTKEYMSTLTYGFNGNV
TGDDTGKIGGLIGANVSIGHTLKYVQPDFKTILESPTDKKVGWKVIFNNM
VNQNWGPYDRDSWNPVYGNQLFMKTRNGSMKAADNFLDPNKASSLLSSGF
SPDFATVITMDRKASKQQTNIDVIYERVRDDYQLHWTSTNWKGTNTKDKW
IDRSSERYKIDWEKEEMTN
SEQ ID NO: 2
MADSDINIKTGTTDIGSNTTVKTGDLVTYDKENGMHKKVFYSFIDDKNHN
KKLLVIRTKGTIAGQYRVYSEEGANKSGLAWPSAFKVQLQLPDNEVAQIS
DYYPRNSIDTKEYMSTLTYGFNGNVTGDDTGKIGGLIGANVSIGHTLKYV
QPDFKTILESPTDKKVGWKVIFNNMVNQNWGPYDRDSWNPVYGNQLFMKT
RNGSMKAADNFLDPNKASSLLSSGFSPDFATVITMDRKASKQQTNIDVIY
ERVRDDYQLHWTSTNWKGTNTKDKWIDRSSERYKIDWEKEEMTN
SEQ ID NO: 3
MNMKKKEKHAIRKKSIGVASVLVGTLIGFGLLSSKEADASENSVTQSDSA
SNESKSNDSSSVSAAPKTDDTNVSDTKTSSNTNNGETSVAQNPAQQETTQ
SSSTNATTEETPVTGEATTTTTNQANTPATTQSSNTNAEELVNQTSNETT
SNDTNTVSSVNSPQNSTNAENVSTTQDTSTEATPSNNESAPQSTDASNKD
VVNQAVNTSAPRMRAFSLAAVAADAPVAGTDITNQLTNVTVGIDSGTTVY
PHQAGYVKLNYGFSVPNSAVKGDTFKITVPKELNLNGVTSTAKVPPIMAG
DQVLANGVIDSDGNVIYTFTDYVNTKDDVKATLTMPAYIDPENVKKTGNV
TLATGIGSTTANKTVLVDYEKYGKFYNLSIKGTIDQIDKTNNTYRQTIYV
NPSGDNVIAPVLTGNLKPNTDSNALIDQQNTSIKVYKVDNAADLSESYFV
NPENFEDVTNSVNITFPNPNQYKVEFNTPDDQITTPYIVVVNGHIDPNSK
GDLALRSTLYGYNSNIIWRSMSWDNEVAFNNGSGSGDGIDKPVVPEQPDE
PGEIEPIPEDSDSDPGSDSGSDSNSDSGSDSGSDSTSDSGSDSASDSDSA
SDSDSASDSDSASDSDSASDSDSDNDSDSDSDSDSDSDSDSDSDSDSDSD
SDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSD
SDSDSDSDSDSDSDSDSASDSDSDSDSDSDSDSDSDSDSDSDSDSDSDSD
SDSDSDSESDSDSDSDSDSDSDSDSDSDSDSASDSDSGSDSDSSSDSDSE
SDSNSDSESVSNNNVVPPNSPKNGTNASNKNEAKDSKEPLPDTGSEDEAN
TSLIWGLLASIGSLLLFRRKKENKDKK
SEQ ID NO: 4
MSENSVTQSDSASNESKSNDSSSVSAAPKTDDTNVSDTKTSSNTNNGETS
VAQNPAQQETTQSSSTNATTEETPVTGEATTTTTNQANTPATTQSSNTNA
EELVNQTSNETTSNDTNTVSSVNSPQNSTNAENVSTTQDTSTEATPSNNE
SAPQSTDASNKDVVNQAVNTSAPRMRAFSLAAVAADAPVAGTDITNQLTN
VTVGIDSGTTVYPHQAGYVKLNYGFSVPNSAVKGDTFKITVPKELNLNGV
TSTAKVPPIMAGDQVLANGVIDSDGNVIYTFTDYVNTKDDVKATLTMPAY
IDPENVKKTGNVTLATGIGSTTANKTVLVDYEKYGKFYNLSIKGTIDQID
KTNNTYRQTIYVNPSGDNVIAPVLTGNLKPNTDSNALIDQQNTSIKVYKV
DNAADLSESYFVNPENFEDVTNSVNITFPNPNQYKVEFNTPDDQITTPYI
VVVNGHIDPNSKGDLALRSTLYGYNSNIIWRSMSWDNEVAFNNGSGSGDG
IDKPVVPEQPDEPGEIEPIPE
SEQ ID NO: 5
MNMKKKEKHAIRKKSIGVASVLVGTLIGFGLLSSKEADASENSVTQSDSA
SNESKSNDSSSVSAAPKTDDTNVSDTKTSSNTNNGETSVAQNPAQQETTQ
SSSTNATTEETPVTGEATTTTTNQANTPATTQSSNTNAEELVNQTSNETT
SNDTNTVSSVNSPQNSTNAENVSTTQDTSTEATPSNNESAPQSTDASNKD
VVNQAVNTSAPRMRAFSLAAVAADAPVAGTDITNQLTNVTVGIDSGTTVY
PHQAGYVKLNYGFSVPNSAVKGDTFKITVPKELNLNGVTSTAKVPPIMAG
DQVLANGVIDSDGNVIYTFTDYVNTKDDVKATLTMPAYIDPENVKKTGNV
TLATGIGSTTANKTVLVDYEKYGKFYNLSIKGTIDQIDKTNNTYRQTIYV
NPSGDNVIAPVLTGNLKPNTDSNALIDQQNTSIKVYKVDNAADLSESYFV
NPENFEDVTNSVNITFPNPNQYKVEFNTPDDQITTPYIVVVNGHIDPNSK
GDLALRSTLYGYNSNIIWRSMSWDNEVAFNNGSGSGDGIDKPVVPEQPDE
PGEIEPIPE
SEQ ID NO: 6
SLAAVAADAPVAGTDITNQLTNVTVGIDSGTTVYPHQAGYVKLNYGFSVP
NSAVKGDTFKITVPKELNLNGVTSTAKVPPIMAGDQVLANGVIDSDGNVI
YTFTDYVNTKDDVKATLTMPAYIDPENVKKTGNVTLATGIGSTTANKTVL
VDYEKYGKFYNLSIKGTIDQIDKTNNTYRQTIYVNPSGDNVIAPVLTGNL
KPNTDSNALIDQQNTSIKVYKVDNAADLSESYFVNPENFEDVTNSVNITF
PNPNQYKVEFNTPDDQITTPYIVVVNGHIDPNSKGDLALRSTLYGYNSNI
IWRSMSWDNEVAFNNGSGSGDGIDKPVVPEQPDEPGEIEPIPE
SEQ ID NO: 7
GTDITNQLTNVTVGIDSGTTVYPHQAGYVKLNYGFSVPNSAVKGDTFKIT
VPKELNLNGVTSTAKVPPIMAGDQVLANGVIDSDGNVIYTFTDYVNTKDD
VKATLTMPAYIDPENVKKTGNVTLATGIGSTTANKTVLVDYEKYGKFYNL
SIKGTIDQIDKTNNTYRQTIYVNPSGDNVIAPVLTGNLKPNTDSNALIDQ
QNTSIKVYKVDNAADLSESYFVNPENFEDVTNSVNITFPNPNQYKVEFNT
PDDQITTPYIVVVNGHIDPNSKGDLALRSTLYGYNSNIIWRSMSWDNEVA
FNNGSGSGDGIDKPVVPEQPDEPGEIEPIPE
SEQ ID NO: 8
MNMKKKEKHAIRKKSIGVASVLVGTLIGFGLLSSKEADASENSVTQSDSA
SNESKSNDSSSVSAAPKTDDTNVSDTKTSSNTNNGETSVAQNPAQQETTQ
SSSTNATTEETPVTGEATTTTTNQANTPATTQSSNTNAEELVNQTSNETT
SNDTNTVSSVNSPQNSTNAENVSTTQDTSTEATPSNNESAPQSTDASNKD
VVNQAVNTSAPRMRAFSLAAVAADAPVAGTDITNQLTNVTVGIDSGTTVY
PHQAGYVKLNYGFSVPNSAVKGDTFKITVPKELNLNGVTSTAKVPPIMAG
DQVLANGVIDSDGNVIYTFTDYVNTKDDVKATLTMSAAIDPENVKKTGNV
TLATGIGSTTANKTVLVDYEKYGKFYNLSIKGTIDQIDKTNNTYRQTIYV
NPSGDNVIAPVLTGNLKPNTDSNALIDQQNTSIKVYKVDNAADLSESYFV
NPENFEDVTNSVNITFPNPNQYKVEFNTPDDQITTPYIVVVNGHIDPNSK
GDLALRSTLYGYNSNIIWRSMSWDNEVAFNNGSGSGDGIDKPVVPEQPDE
PGEIEPIPEDSDSDPGSDSGSDSNSDSGSDSGSDSTSDSGSDSASDSDSA
SDSDSASDSDSASDSDSASDSDSDNDSDSDSDSDSDSDSDSDSDSDSDSD
SDSDSDSDSDSDSDSDSDSDSDSDSDSDSDSDSDSDSDSDSDSDSDSDSD
SDSDSDSDSDSDSDSDSDSDSDSDSDSDSDSDSDSDSDSDSDSDSDSDSD
SDSDSDSDSDSDSDSDSASDSDSDSDSDSDSDSDSDSDSDSDSDSDSDSD
SDSDSDSESDSDSDSDSDSDSDSDSDSDSDSASDSDSGSDSDSSSDSDSE
SDSNSDSESVSNNNVVPPNSPKNGTNASNKNEAKDSKEPLPDTGSEDEAN
TSLIWGLLASIGSLLLFRRKKENKDKK
SEQ ID NO: 9
MSENSVTQSDSASNESKSNDSSSVSAAPKTDDTNVSDTKTSSNTNNGETS
VAQNPAQQETTQSSSTNATTEETPVTGEATTTTTNQANTPATTQSSNTNA
EELVNQTSNETTSNDTNTVSSVNSPQNSTNAENVSTTQDTSTEATPSNNE
SAPQSTDASNKDVVNQAVNTSAPRMRAFSLAAVAADAPVAGTDITNQLTN
VTVGIDSGTTVYPHQAGYVKLNYGFSVPNSAVKGDTFKITVPKELNLNGV
TSTAKVPPIMAGDQVLANGVIDSDGNVIYTFTDYVNTKDDVKATLTMSAA
IDPENVKKTGNVTLATGIGSTTANKTVLVDYEKYGKFYNLSIKGTIDQID
KTNNTYRQTIYVNPSGDNVIAPVLTGNLKPNTDSNALIDQQNTSIKVYKV
DNAADLSESYFVNPENFEDVTNSVNITFPNPNQYKVEFNTPDDQITTPYI
VVVNGHIDPNSKGDLALRSTLYGYNSNIIWRSMSWDNEVAFNNGSGSGDG
IDKPVVPEQPDEPGEIEPIPE
SEQ ID NO: 10
MNMKKKEKHAIRKKSIGVASVLVGTLIGFGLLSSKEADASENSVTQSDSA
SNESKSNDSSSVSAAPKTDDTNVSDTKTSSNTNNGETSVAQNPAQQETTQ
SSSTNATTEETPVTGEATTTTTNQANTPATTQSSNTNAEELVNQTSNETT
SNDTNTVSSVNSPQNSTNAENVSTTQDTSTEATPSNNESAPQSTDASNKD
VVNQAVNTSAPRMRAFSLAAVAADAPVAGTDITNQLTNVTVGIDSGTTVY
PHQAGYVKLNYGFSVPNSAVKGDTFKITVPKELNLNGVTSTAKVPPIMAG
DQVLANGVIDSDGNVIYTFTDYVNTKDDVKATLTMSAAIDPENVKKTGNV
TLATGIGSTTANKTVLVDYEKYGKFYNLSIKGTIDQIDKTNNTYRQTIYV
NPSGDNVIAPVLTGNLKPNTDSNALIDQQNTSIKVYKVDNAADLSESYFV
NPENFEDVTNSVNITFPNPNQYKVEFNTPDDQITTPYIVVVNGHIDPNSK
GDLALRSTLYGYNSNIIWRSMSWDNEVAFNNGSGSGDGIDKPVVPEQPDE
PGEIEPIPE
SEQ ID NO: 11
SLAAVAADAPVAGTDITNQLTNVTVGIDSGTTVYPHQAGYVKLNYGFSVP
NSAVKGDTFKITVPKELNLNGVTSTAKVPPIMAGDQVLANGVIDSDGNVI
YTFTDYVNTKDDVKATLTMSAAIDPENVKKTGNVTLATGIGSTTANKTVL
VDYEKYGKFYNLSIKGTIDQIDKTNNTYRQTIYVNPSGDNVIAPVLTGNL
KPNTDSNALIDQQNTSIKVYKVDNAADLSESYFVNPENFEDVTNSVNITF
PNPNQYKVEFNTPDDQITTPYIVVVNGHIDPNSKGDLALRSTLYGYNSNI
IWRSMSWDNEVAFNNGSGSGDGIDKPVVPEQPDEPGEIEPIPE
SEQ ID NO: 12
GTDITNQLTNVTVGIDSGTTVYPHQAGYVKLNYGFSVPNSAVKGDTFKIT
VPKELNLNGVTSTAKVPPIMAGDQVLANGVIDSDGNVIYTFTDYVNTKDD
VKATLTMSAAIDPENVKKTGNVTLATGIGSTTANKTVLVDYEKYGKFYNL
SIKGTIDQIDKTNNTYRQTIYVNPSGDNVIAPVLTGNLKPNTDSNALIDQ
QNTSIKVYKVDNAADLSESYFVNPENFEDVTNSVNITFPNPNQYKVEFNT
PDDQITTPYIVVVNGHIDPNSKGDLALRSTLYGYNSNIIWRSMSWDNEVA
FNNGSGSGDGIDKPVVPEQPDEPGEIEPIPE
SEQ ID NO: 13
MKTRIVSSVTTTLLLGSILMNPVANAADSDINIKTGTTDIGSNTTVKTGD
LVTYDKENGMRKKVFYSFIDDKNHNKKLLVIRTKGTIAGQYRVYSEEGAN
KSGLAWPSAFKVQLQLPDNEVAQISDYYPRNSIDTKEYMSTLTYGFNGNV
TGDDTGKIGGLIGANVSIGHTLKYVQPDFKTILESPTDKKVGWKVIFNNM
VNQNWGPYDRDSWNPVYGNQLFMKTRNGSMKAADNFLDPNKASSLLSSGF
SPDFATVITMDRKASKQQTNIDVIYERVRDDYQLHWTSTNWKGTNTKDKW
IDRSSERYKIDWEKEEMTN
SEQ ID NO: 14
MADSDINIKTGTTDIGSNTTVKTGDLVTYDKENGMRKKVFYSFIDDKNHN
KKLLVIRTKGTIAGQYRVYSEEGANKSGLAWPSAFKVQLQLPDNEVAQIS
DYYPRNSIDTKEYMSTLTYGFNGNVTGDDTGKIGGLIGANVSIGHTLKYV
QPDFKTILESPTDKKVGWKVIFNNMVNQNWGPYDRDSWNPVYGNQLFMKT
RNGSMKAADNFLDPNKASSLLSSGFSPDFATVITMDRKASKQQTNIDVIY
ERVRDDYQLHWTSTNWKGTNTKDKWIDRSSERYKIDWEKEEMTN
SEQ ID NO: 15
MASLAAVAADAPVAGTDITNQLTNVTVGIDSGTTVYPHQAGYVKLNYGFS
VPNSAVKGDTFKITVPKELNLNGVTSTAKVPPIMAGDQVLANGVIDSDGN
VIYTFTDYVNTKDDVKATLTMPAYIDPENVKKTGNVTLATGIGSTTANKT
VLVDYEKYGKFYNLSIKGTIDQIDKTNNTYRQTIYVNPSGDNVIAPVLTG
NLKPNTDSNALIDQQNTSIKVYKVDNAADLSESYFVNPENFEDVTNSVNI
TFPNPNQYKVEFNTPDDQITTPYIVVVNGHIDPNSKGDLALRSTLYGYNS
NIIWRSMSWDNEVAFNNGSGSGDGIDKPVVPEQPDEPGEIEPIPE
SEQ ID NO: 16
MAGTDITNQLTNVTVGIDSGTTVYPHQAGYVKLNYGFSVPNSAVKGDTFK
ITVPKELNLNGVTSTAKVPPIMAGDQVLANGVIDSDGNVIYTFTDYVNTK
DDVKATLTMPAYIDPENVKKTGNVTLATGIGSTTANKTVLVDYEKYGKFY
NLSIKGTIDQIDKTNNTYRQTIYVNPSGDNVIAPVLTGNLKPNTDSNALI
DQQNTSIKVYKVDNAADLSESYFVNPENFEDVTNSVNITFPNPNQYKVEF
NTPDDQITTPYIVVVNGHIDPNSKGDLALRSTLYGYNSNIIWRSMSWDNE
VAFNNGSGSGDGIDKPVVPEQPDEPGEIEPIPE
SEQ ID NO: 17
MASLAAVAADAPVAGTDITNQLTNVTVGIDSGTTVYPHQAGYVKLNYGFS
VPNSAVKGDTFKITVPKELNLNGVTSTAKVPPIMAGDQVLANGVIDSDGN
VIYTFTDYVNTKDDVKATLTMSAAIDPENVKKTGNVTLATGIGSTTANKT
VLVDYEKYGKFYNLSIKGTIDQIDKTNNTYRQTIYVNPSGDNVIAPVLTG
NLKPNTDSNALIDQQNTSIKVYKVDNAADLSESYFVNPENFEDVTNSVNI
TFPNPNQYKVEFNTPDDQITTPYIVVVNGHIDPNSKGDLALRSTLYGYNS
NIIWRSMSWDNEVAFNNGSGSGDGIDKPVVPEQPDEPGEIEPIPE
SEQ ID NO: 18
MAGTDITNQLTNVTVGIDSGTTVYPHQAGYVKLNYGFSVPNSAVKGDTFK
ITVPKELNLNGVTSTAKVPPIMAGDQVLANGVIDSDGNVIYTFTDYVNTK
DDVKATLTMSAAIDPENVKKTGNVTLATGIGSTTANKTVLVDYEKYGKFY
NLSIKGTIDQIDKTNNTYRQTIYVNPSGDNVIAPVLTGNLKPNTDSNALI
DQQNTSIKVYKVDNAADLSESYFVNPENFEDVTNSVNITFPNPNQYKVEF
NTPDDQITTPYIVVVNGHIDPNSKGDLALRSTLYGYNSNIIWRSMSWDNE
VAFNNGSGSGDGIDKPVVPEQPDEPGEIEPIPE

### Example 2 Preparation of Vaccine Components

A four component staphylococcal vaccine was prepared which contained S. aureus Type 5 capsular polysaccharide conjugated to a tetanus toxoid carrier protein, S. aureus Type 8 capsular polysaccharide conjugated to a tetanus toxoid carrier protein, a fragment of ClfA containing the N2 and N3 domains and point mutations at residues 336 and 338 in which P336 is changed to serine and Y338 is changed to alanine, and alpha toxoid which is detoxified by a point mutation at residue 35 with H35 being changed to arginine. The capsular polysaccharides were conjugated to tetanus toxoid using CDAP as the coupling agent. This conjugation process is described in WO 07/113222.

Four formulations of the staphylococcal vaccine were made:

5/10 contained: 5 μg saccharide dose of Type 5—tetanus toxoid conjugate, 5 μg saccharide dose of Type 8—tetanus toxoid conjugate, 10 μg of alpha toxoid and 10 μg of the ClfA truncate described above.
10/30 contained: 10 μg saccharide dose of Type 5—tetanus toxoid conjugate, 10 μg saccharide dose of Type 8—tetanus toxoid conjugate, 30 μg of alpha toxoid and 30 μg of the ClfA truncate described above.
5/10AS contained: 5 μg saccharide dose of Type 5—tetanus toxoid conjugate, 5 μg saccharide dose of Type 8—tetanus toxoid conjugate, 10 μg of alpha toxoid and 10 μg of the ClfA truncate described above, adjuvanted with an oil in water elusion containing squalene, alpha-tocopherol and polyoxyethylene sorbitan monooleate.
10/30AS contained: 10 μg saccharide dose of Type 5—tetanus toxoid conjugate, 10 μg saccharide dose of Type 8—tetanus toxoid conjugate, 30 μg of alpha toxoid and 30 μg of the ClfA truncate described above, adjuvanted with an oil in water elusion containing squalene, alpha-tocopherol and polyoxyethylene sorbitan monooleate.

### Example 3 Clinical Trial Results Using the 4 Component Staphylococcal Vaccine

A phase I clinical trial was carried out using a total of 88 healthy adults from 18 to 40 years old. The control group contained 30 subjects who were inoculated with saline. The remaining subjects were divided into four arms with 15/14 subjects being immunised with each of the formulations described in example 2 (5/10, 5/10AS, 10/30 and 10/30AS). Vaccine doses were given at the start of the trial and after one month and at six months. Blood samples for humoral analysis were taken at day 0, 7, 14 and 30 after each dose and at day 360 and 540.

Details of the subjects are provided below.

| Group | N | Mean Age | % female |
|---|---|---|---|
| 5/10 | 15 | 31.1 | 73.3 |
| 5/10AS | 15 | 31.9 | 33.3 |
| 10/30 | 14 | 30.9 | 42.9 |
| 10/30AS | 14 | 30.6 | 50 |
| Saline | 30 | 30.1 | 50 |

### Reactogenicity and Safety

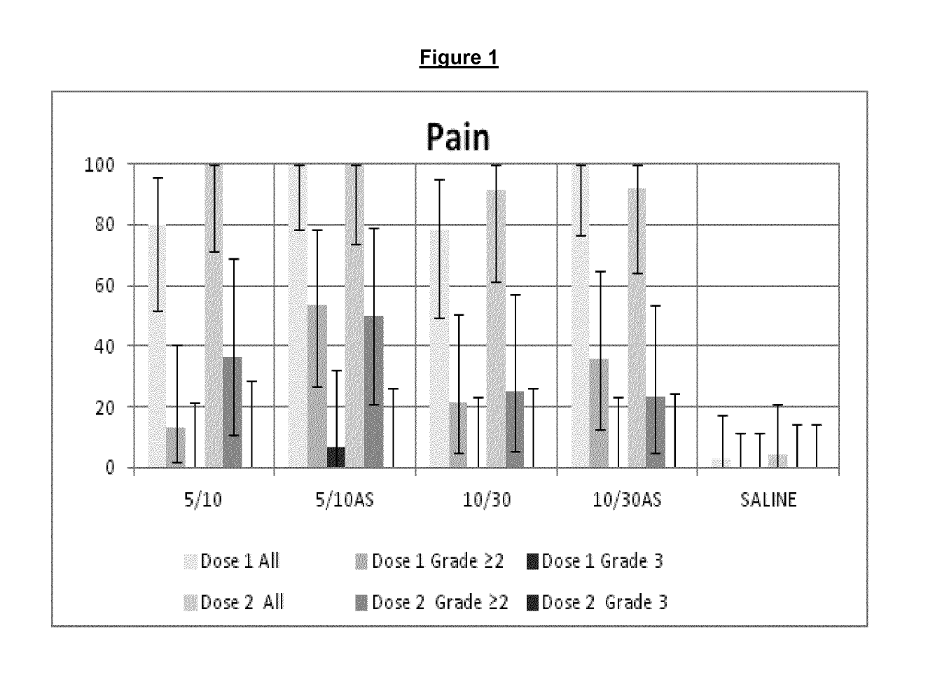

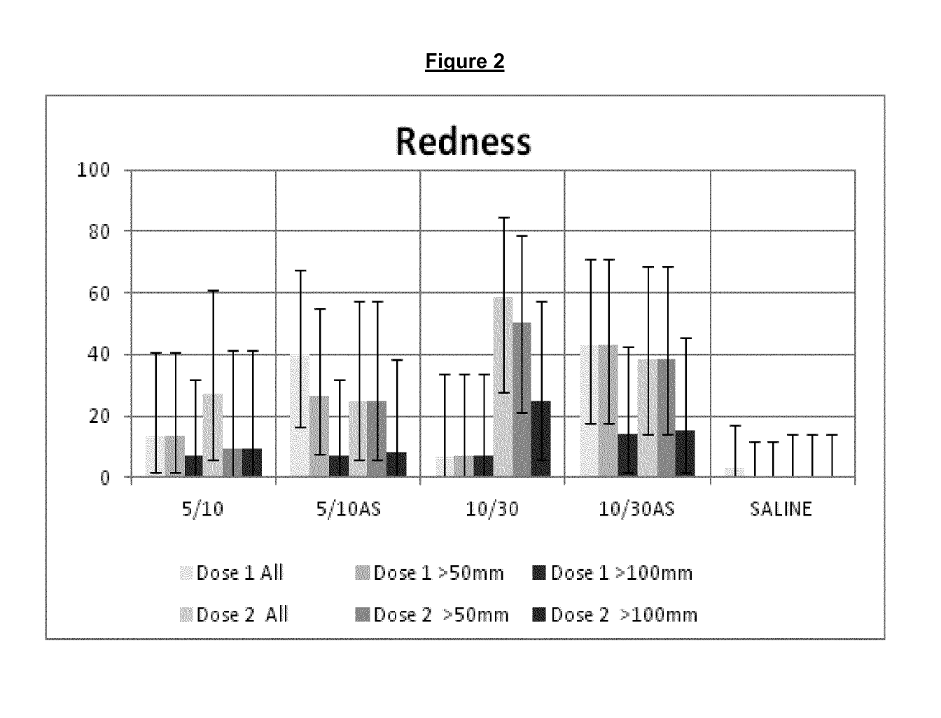

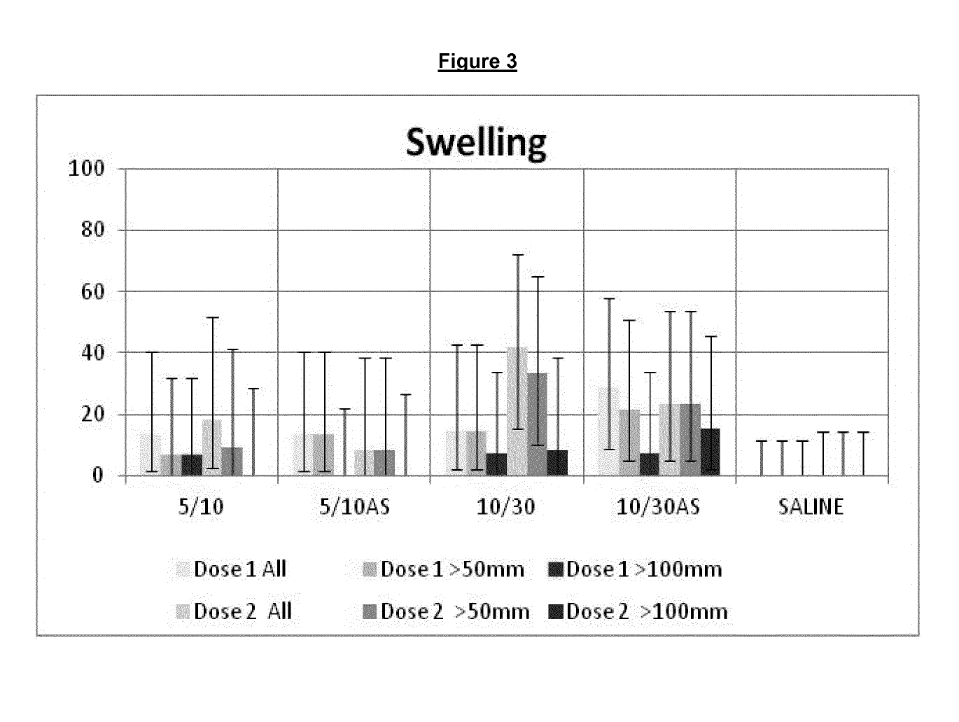

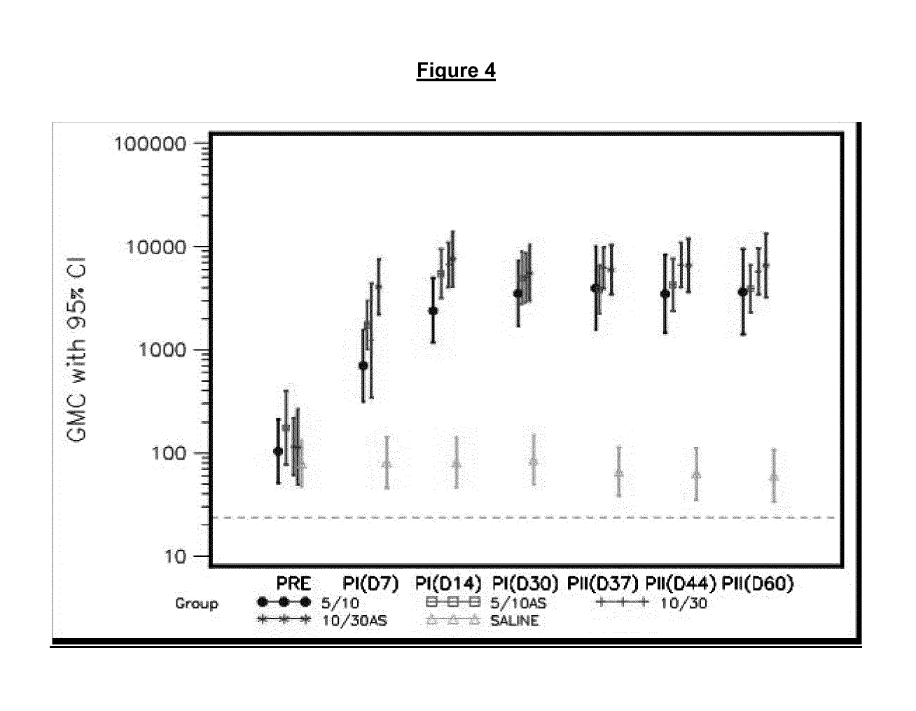

The 4 component staphylococcal vaccine was generally safe and well tolerated. After the first and second doses no serious adverse events and no potential immune mediated disorders were observed. The percentage of subjects reporting pain, redness and swelling after dose 1 and dose 2 is shown in FIGS. 1-3. Pain was experienced at the injection site in 78.6-100% of subjects in the vaccine groups compared to 3-4% in the control group (see FIG. 1). However, only one case was graded 3. Results for the incidence of redness and swelling are shown in FIGS. 2 and 3. For both parameters, there was a trend for a higher incidence of redness/swelling following administration of the second dose compared to after a single dose for the 10/30 arm of the study.

### Immunogenicity

Blood samples taken from subjects on day 0 and 7, 14 and 30 days following the first second and third immunisations were tested by Luminex or ELISA to establish the level of IgG produced against each antigen of the four component staphylococcal vaccine.

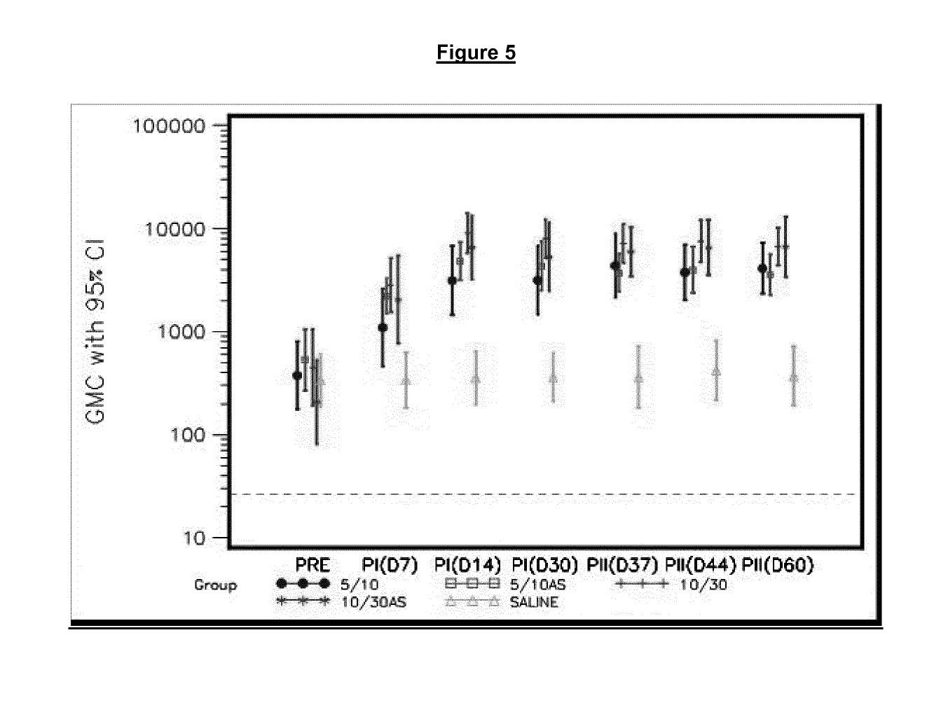

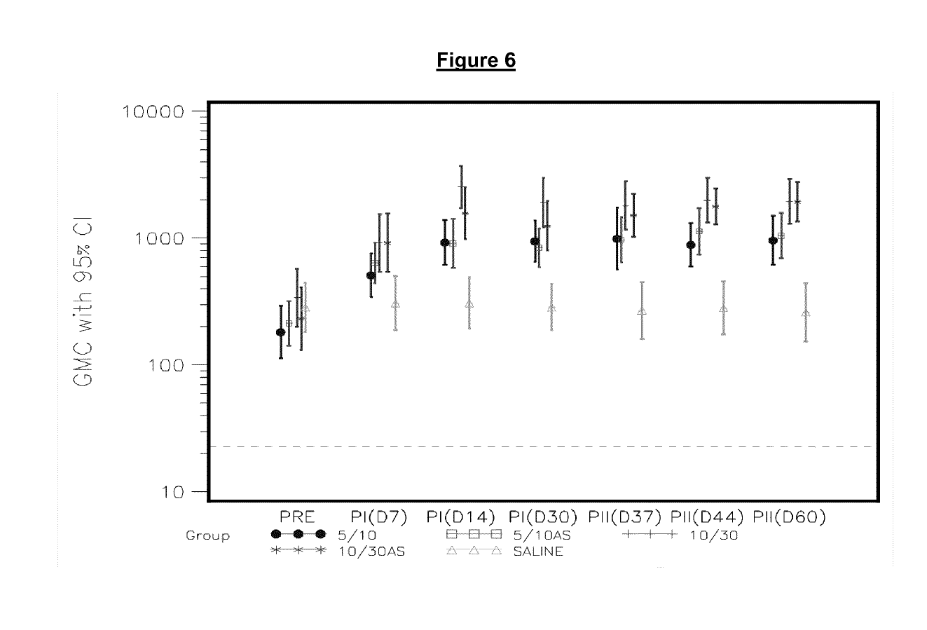

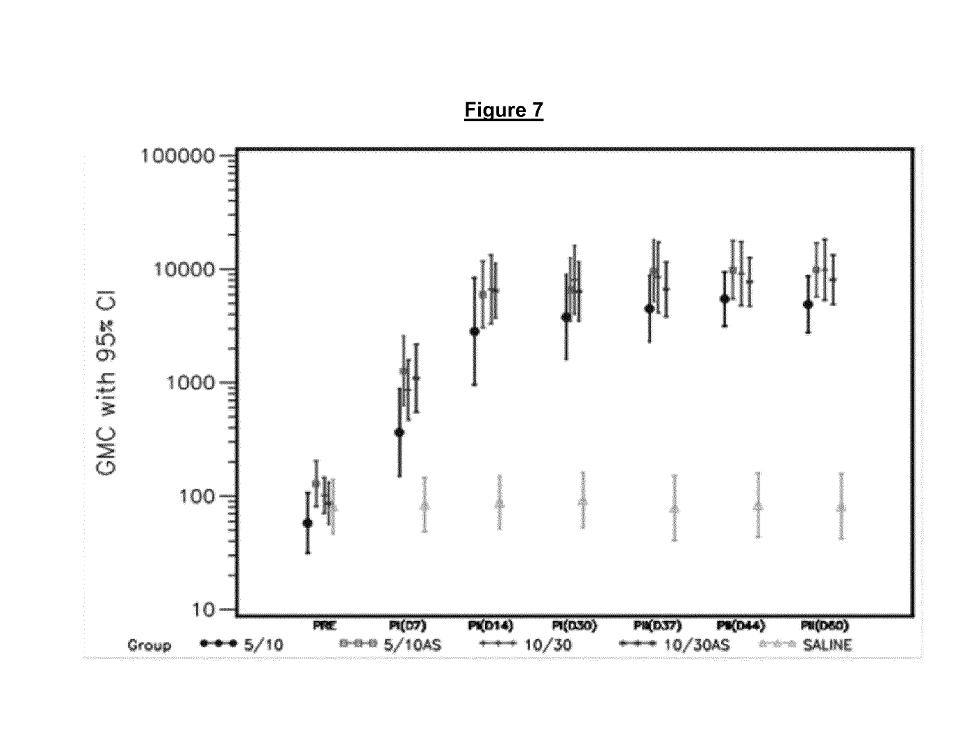

Results for immunogenicity are shown in FIGS. 4-8 and in the Tables 1-5 below.

Prevaccination, there was 83.3-100% seropositivity for all assays. Despite considerable levels of background immunity, the 4 component vaccine was able to elicit a robust immune response against all 4 components.

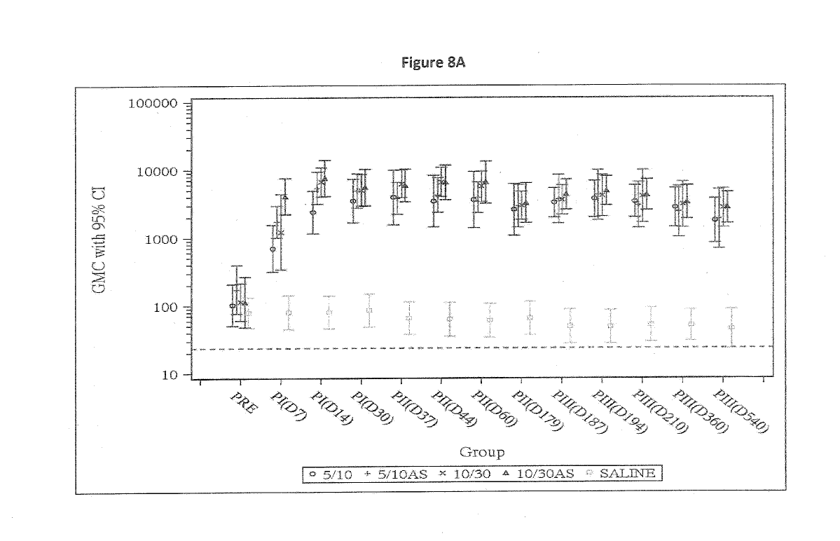

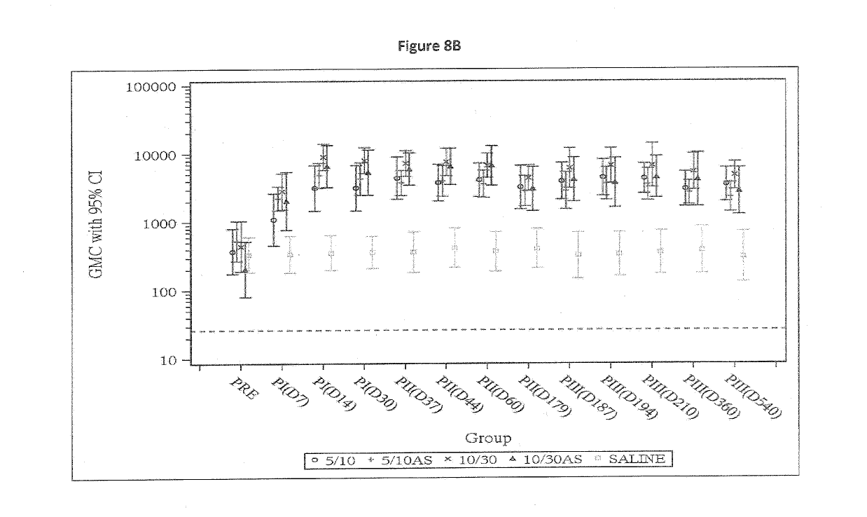

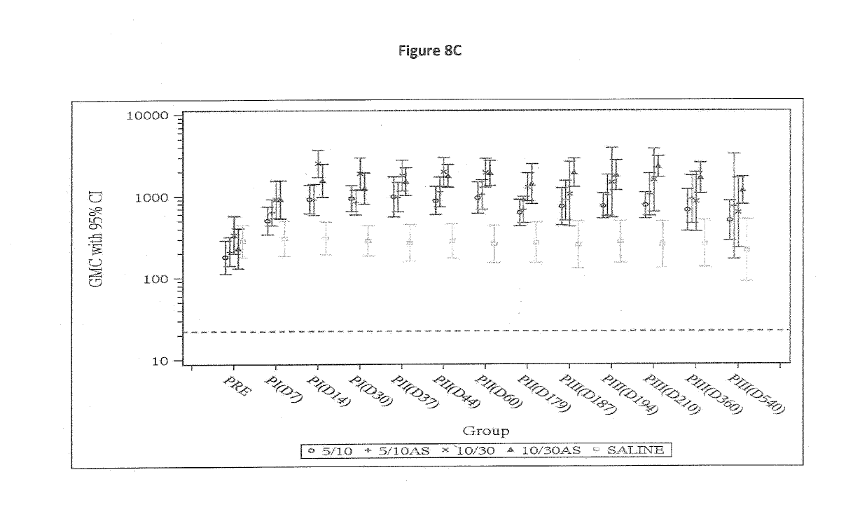

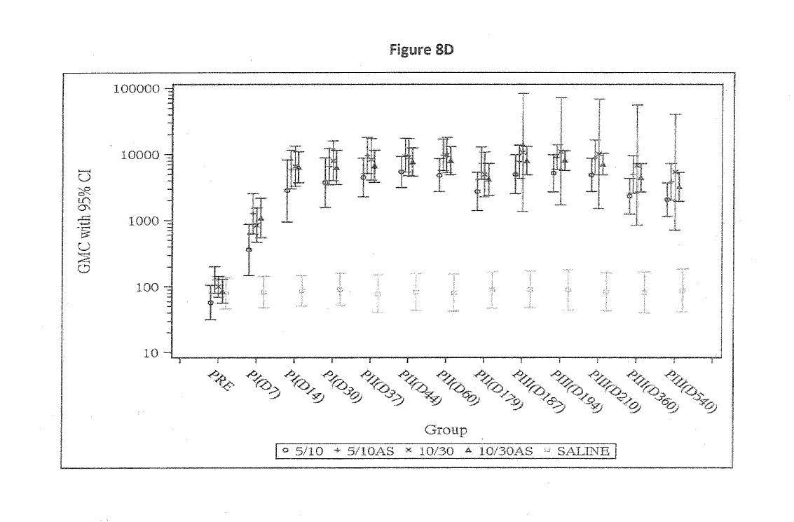

FIGS. 4-7 show that for CPS5, CPS8, alpha toxoid and ClfA, the first immunisation produced the largest increase in immunogenicity with strong increases of GMC being apparent at day 14 and 30. The second immunisation on day 30 did not produce a further increase in immunogenicity and GMC levels remain at a similar level between days 30 and 60. FIG. 8 shows that the third immunisation after 6 months did not provoke a further increase in GMC with GMC levels remaining approximately the same for the four components between day 30 and day 540. A single immunisation is therefore an efficient way of producing a maximal immune response.

The immunogenicity results for the 10/30 dosage appear to be stronger than for the 5/10 dosage with an approximately 1-5-2 fold increase of GMC for CPS5, CPS8 and alpha toxoid. In the case of ClfA the increase in GMC was about 3.8 fold at the higher dose. The addition of oil in water emulsion adjuvant did not increase the immunogenicity of the 4 component vaccine as demonstrated by a comparison of antibody response elicited by the 5/10 and 5/10AS arms and the 10/30 and 10/30AS arms.

TABLE 1
Seropositivity rates and GMCs for Staph aureus.CPS
5 Ab.IgG antibodies (ATP cohort for immunogenicity)

| | | | | ≧23.6 LU/ml | | | | GMC | | |
|---|---|---|---|---|---|---|---|---|---|---|
| | | | | | | 95% CI | | | 95% CI | |
| Antibody | Group | Timing | N | n | % | LL | UL | value | LL | UL |
| Staph aureus.CPS 5 | 5/10 | PRE | 15 | 13 | 86.7 | 59.5 | 98.3 | 104.00 | 51.24 | 211.07 |
| Ab.IgG | | PI(D7) | 15 | 14 | 93.3 | 68.1 | 99.8 | 702.89 | 316.09 | 1562.98 |
| | | PI(D14) | 11 | 11 | 100 | 71.5 | 100 | 2393.81 | 1164.68 | 4920.09 |
| | | PI(D30) | 14 | 14 | 100 | 76.8 | 100 | 3515.50 | 1690.01 | 7312.81 |
| | | PII(D37) | 9 | 9 | 100 | 66.4 | 100 | 3970.84 | 1570.67 | 10038.80 |
| | | PII(D44) | 9 | 9 | 100 | 66.4 | 100 | 3485.16 | 1456.13 | 8341.54 |
| | | PII(D60) | 9 | 9 | 100 | 66.4 | 100 | 3648.17 | 1414.59 | 9408.46 |
| | 5/10AS | PRE | 15 | 14 | 93.3 | 68.1 | 99.8 | 175.35 | 77.12 | 398.69 |
| | | PI(D7) | 15 | 15 | 100 | 78.2 | 100 | 1745.15 | 1016.89 | 2994.97 |
| | | PI(D14) | 15 | 15 | 100 | 78.2 | 100 | 5447.98 | 3150.01 | 9422.35 |
| | | PI(D30) | 15 | 15 | 100 | 78.2 | 100 | 4962.11 | 2766.72 | 8899.55 |
| | | PII(D37) | 12 | 12 | 100 | 73.5 | 100 | 3831.22 | 2234.21 | 6569.79 |
| | | PII(D44) | 12 | 12 | 100 | 73.5 | 100 | 4262.74 | 2373.12 | 7656.98 |
| | | PII(D60) | 12 | 12 | 100 | 73.5 | 100 | 3920.80 | 2316.00 | 6637.61 |
| | 10/30 | PRE | 14 | 14 | 100 | 76.8 | 100 | 114.74 | 60.89 | 216.23 |
| | | PI(D7) | 6 | 6 | 100 | 54.1 | 100 | 1231.04 | 342.92 | 4419.30 |
| | | PI(D14) | 11 | 11 | 100 | 71.5 | 100 | 6684.54 | 4060.86 | 11003.35 |
| | | PI(D30) | 14 | 14 | 100 | 76.8 | 100 | 5023.61 | 2922.27 | 8636.00 |
| | | PII(D37) | 12 | 12 | 100 | 73.5 | 100 | 6228.11 | 3904.47 | 9934.61 |
| | | PII(D44) | 12 | 12 | 100 | 73.5 | 100 | 6625.99 | 4026.07 | 10904.85 |
| | | PII(D60) | 12 | 12 | 100 | 73.5 | 100 | 5749.41 | 3442.63 | 9601.86 |
| | 10/30AS | PRE | 14 | 13 | 92.9 | 66.1 | 99.8 | 114.02 | 48.87 | 266.02 |
| | | PI(D7) | 6 | 6 | 100 | 54.1 | 100 | 4088.58 | 2215.34 | 7545.81 |
| | | PI(D14) | 11 | 11 | 100 | 71.5 | 100 | 7598.72 | 4120.90 | 14011.61 |
| | | PI(D30) | 14 | 14 | 100 | 76.8 | 100 | 5569.08 | 2994.06 | 10358.73 |
| | | PII(D37) | 13 | 13 | 100 | 75.3 | 100 | 5930.99 | 3425.26 | 10269.76 |
| | | PII(D44) | 13 | 13 | 100 | 75.3 | 100 | 6588.83 | 3645.17 | 11909.64 |
| | | PII(D60) | 13 | 13 | 100 | 75.3 | 100 | 6582.67 | 3229.11 | 13419.03 |
| | SALINE | PRE | 30 | 25 | 83.3 | 65.3 | 94.4 | 79.19 | 46.96 | 133.54 |
| | | PI(D7) | 29 | 23 | 79.3 | 60.3 | 92.0 | 80.62 | 45.45 | 143.00 |
| | | PI(D14) | 29 | 23 | 79.3 | 60.3 | 92.0 | 80.57 | 46.22 | 140.43 |
| | | PI(D30) | 30 | 24 | 80.0 | 61.4 | 92.3 | 85.65 | 49.13 | 149.31 |
| | | PII(D37) | 24 | 20 | 83.3 | 62.6 | 95.3 | 65.60 | 38.22 | 112.60 |
| | | PII(D44) | 23 | 19 | 82.6 | 61.2 | 95.0 | 62.84 | 35.17 | 112.30 |
| | | PII(D60) | 24 | 18 | 75.0 | 53.3 | 90.2 | 60.24 | 33.89 | 107.06 |

5/10 = 5 μg CPS5-TT, 5 μg CPS8-TT, 10 μg ClfA, 10 μg α-toxoid
5/10AS = 5 μg CPS5-TT, 5 μg CPS8-TT, 10 μg ClfA, 10 μg α-toxoid adjuvanted with AS03B
10/30 = 10 μg CPS5-TT, 10 μg CPS8-TT, 30 μg ClfA, 30 μg α-toxoid
10/30AS = 10 μg CPS5-TT, 10 μg CPS8-TT, 30 μg ClfA, 30 μg α-toxoid adjuvanted with AS03B
SALINE = pooling of SALINE1 and SALINE2
GMC = geometric mean antibody concentration calculated on all subjects
N = number of subjects with available results
n/% = number/percentage of subjects with concentration within the specified range
95% CI = 95% confidence interval;
LL = Lower Limit,
UL = Upper Limit
PRE = pre dose 1
PI(D7) = 7 days post dose 1
PI(D14) = 14 days post dose 1
PI(D30) = 30 days post dose 1 (blood sample taken at Visits 5 or 6)
PII(D37) = 7 days post dose 2
PII(D44) = 14 days post dose 2
PII(D60) = 30 days post dose 2

TABLE 1
Seropositivity rates and GMCs for Staph aureus.CPS
8 Ab.IgG antibodies (ATP cohort for immunogenicity)

| | | | | ≧26.5 LU/ml | | | | GMC | | |
|---|---|---|---|---|---|---|---|---|---|---|
| | | | | | | 95% CI | | | 95% CI | |
| Antibody | Group | Timing | N | n | % | LL | UL | value | LL | UL |
| Staph aureus.CPS 8 | 5/10 | PRE | 15 | 14 | 93.3 | 68.1 | 99.8 | 377.47 | 176.95 | 805.24 |
| Ab.IgG | | PI(D7) | 15 | 15 | 100 | 78.2 | 100 | 1101.09 | 460.23 | 2634.33 |
| | | PI(D14) | 14 | 14 | 100 | 76.8 | 100 | 3151.09 | 1460.34 | 6799.36 |
| | | PI(D30) | 15 | 15 | 100 | 78.2 | 100 | 3169.43 | 1471.27 | 6827.61 |
| | | PII(D37) | 10 | 10 | 100 | 69.2 | 100 | 4382.17 | 2147.01 | 8944.26 |
| | | PII(D44) | 10 | 10 | 100 | 69.2 | 100 | 3776.90 | 2035.45 | 7008.27 |
| | | PII(D60) | 10 | 10 | 100 | 69.2 | 100 | 4120.46 | 2329.69 | 7287.77 |
| | 5/10AS | PRE | 15 | 15 | 100 | 78.2 | 100 | 533.66 | 270.37 | 1053.36 |
| | | PI(D7) | 15 | 15 | 100 | 78.2 | 100 | 2220.14 | 1489.78 | 3308.56 |
| | | PI(D14) | 13 | 13 | 100 | 75.3 | 100 | 4831.66 | 3164.57 | 7376.97 |
| | | PI(D30) | 13 | 13 | 100 | 75.3 | 100 | 4328.02 | 2494.84 | 7508.20 |
| | | PII(D37) | 11 | 11 | 100 | 71.5 | 100 | 3722.46 | 2425.65 | 5712.58 |
| | | PII(D44) | 11 | 11 | 100 | 71.5 | 100 | 3973.72 | 2364.01 | 6679.54 |
| | | PII(D60) | 11 | 11 | 100 | 71.5 | 100 | 3573.72 | 2256.18 | 5660.67 |
| | 10/30 | PRE | 12 | 12 | 100 | 73.5 | 100 | 446.48 | 189.79 | 1050.34 |
| | | PI(D7) | 12 | 12 | 100 | 73.5 | 100 | 2830.32 | 1540.49 | 5200.12 |
| | | PI(D14) | 14 | 14 | 100 | 76.8 | 100 | 9038.91 | 5796.13 | 14095.93 |
| | | PI(D30) | 13 | 13 | 100 | 75.3 | 100 | 7980.64 | 5159.87 | 12343.44 |
| | | PII(D37) | 12 | 12 | 100 | 73.5 | 100 | 7205.23 | 4676.27 | 11101.87 |
| | | PII(D44) | 12 | 12 | 100 | 73.5 | 100 | 7549.64 | 4717.98 | 12080.83 |
| | | PII(D60) | 11 | 11 | 100 | 71.5 | 100 | 6728.09 | 4425.54 | 10228.61 |
| | 10/30AS | PRE | 14 | 12 | 85.7 | 57.2 | 98.2 | 207.57 | 81.34 | 529.65 |
| | | PI(D7) | 11 | 11 | 100 | 71.5 | 100 | 2049.03 | 769.73 | 5454.51 |
| | | PI(D14) | 12 | 12 | 100 | 73.5 | 100 | 6569.22 | 3215.77 | 13419.68 |
| | | PI(D30) | 13 | 13 | 100 | 75.3 | 100 | 5307.09 | 2468.17 | 11411.40 |
| | | PII(D37) | 13 | 13 | 100 | 75.3 | 100 | 5984.18 | 3461.54 | 10345.20 |
| | | PII(D44) | 12 | 12 | 100 | 73.5 | 100 | 6549.44 | 3543.91 | 12103.91 |
| | | PII(D60) | 12 | 12 | 100 | 73.5 | 100 | 6665.14 | 3418.24 | 12996.20 |
| | SALINE | PRE | 28 | 26 | 92.9 | 76.5 | 99.1 | 335.46 | 184.17 | 611.03 |
| | | PI(D7) | 27 | 25 | 92.6 | 75.7 | 99.1 | 340.15 | 182.56 | 633.77 |
| | | PI(D14) | 28 | 26 | 92.9 | 76.5 | 99.1 | 355.41 | 195.74 | 645.30 |
| | | PI(D30) | 30 | 29 | 96.7 | 82.8 | 99.9 | 362.15 | 210.58 | 622.79 |
| | | PII(D37) | 24 | 22 | 91.7 | 73.0 | 99.0 | 361.33 | 182.65 | 714.82 |
| | | PII(D44) | 23 | 22 | 95.7 | 78.1 | 99.9 | 418.45 | 216.00 | 810.66 |
| | | PII(D60) | 24 | 23 | 95.8 | 78.9 | 99.9 | 368.24 | 189.56 | 715.34 |

5/10 = 5 μg CPS5-TT, 5 μg CPS8-TT, 10 μg ClfA, 10 μg α-toxoid
5/10AS = 5 μg CPS5-TT, 5 μg CPS8-TT, 10 μg ClfA, 10 μg α-toxoid adjuvanted with AS03B
10/30 = 10 μg CPS5-TT, 10 μg CPS8-TT, 30 μg ClfA, 30 μg α-toxoid
10/30AS = 10 μg CPS5-TT, 10 μg CPS8-TT, 30 μg ClfA, 30 μg α-toxoid adjuvanted with AS03B
SALINE = pooling of SALINE1 and SALINE2
GMC = geometric mean antibody concentration calculated on all subjects
N = number of subjects with available results
n/% = number/percentage of subjects with concentration within the specified range
95% CI = 95% confidence interval;
LL = Lower Limit,
UL = Upper Limit
PRE = pre dose 1
PI(D7) = 7 days post dose 1
PI(D14) = 14 days post dose 1
PI(D30) = 30 days post dose 1 (blood sample taken at Visits 5 or 6)
PII(D37) = 7 days post dose 2
PII(D44) = 14 days post dose 2
PII(D60) = 30 days post dose 2

TABLE 2
Seropositivity rates and GMCs for Staph aureus alphα-toxin
Ab.IgG antibodies (ATP cohort for immunogenicity)

| | | | | ≧22.5 LU/ml | | | | GMC | | |
|---|---|---|---|---|---|---|---|---|---|---|
| | | | | | | 95% CI | | | 95% CI | |
| Antibody | Group | Timing | N | n | % | LL | UL | value | LL | UL |
| Staph aureus | 5/10 | PRE | 15 | 15 | 100 | 78.2 | 100 | 181.59 | 112.62 | 292.80 |
| alphα-toxin Ab.IgG | | PI(D7) | 15 | 15 | 100 | 78.2 | 100 | 508.56 | 342.65 | 754.79 |
| | | PI(D14) | 14 | 14 | 100 | 76.8 | 100 | 924.97 | 617.24 | 1386.12 |
| | | PI(D30) | 15 | 15 | 100 | 78.2 | 100 | 946.86 | 654.84 | 1369.11 |
| | | PII(D37) | 10 | 10 | 100 | 69.2 | 100 | 991.85 | 565.44 | 1739.82 |
| | | PII(D44) | 10 | 10 | 100 | 69.2 | 100 | 885.68 | 595.57 | 1317.12 |
| | | PII(D60) | 10 | 10 | 100 | 69.2 | 100 | 960.49 | 615.68 | 1498.42 |
| | 5/10AS | PRE | 15 | 15 | 100 | 78.2 | 100 | 212.93 | 142.13 | 318.99 |
| | | PI(D7) | 15 | 15 | 100 | 78.2 | 100 | 639.16 | 441.46 | 925.40 |
| | | PI(D14) | 15 | 15 | 100 | 78.2 | 100 | 910.41 | 586.44 | 1413.34 |
| | | PI(D30) | 15 | 15 | 100 | 78.2 | 100 | 842.98 | 594.48 | 1195.38 |
| | | PII(D37) | 12 | 12 | 100 | 73.5 | 100 | 974.08 | 644.36 | 1472.52 |
| | | PII(D44) | 12 | 12 | 100 | 73.5 | 100 | 1134.60 | 745.18 | 1727.54 |
| | | PII(D60) | 12 | 12 | 100 | 73.5 | 100 | 1048.51 | 693.13 | 1586.12 |
| | 10/30 | PRE | 11 | 11 | 100 | 71.5 | 100 | 339.09 | 200.20 | 574.32 |
| | | PI(D7) | 13 | 13 | 100 | 75.3 | 100 | 919.07 | 543.30 | 1554.74 |
| | | PI(D14) | 13 | 13 | 100 | 75.3 | 100 | 2534.87 | 1728.09 | 3718.31 |
| | | PI(D30) | 14 | 14 | 100 | 76.8 | 100 | 1913.52 | 1224.06 | 2991.33 |
| | | PII(D37) | 12 | 12 | 100 | 73.5 | 100 | 1804.43 | 1163.54 | 2798.33 |
| | | PII(D44) | 12 | 12 | 100 | 73.5 | 100 | 1988.02 | 1326.61 | 2979.21 |
| | | PII(D60) | 12 | 12 | 100 | 73.5 | 100 | 1947.83 | 1295.34 | 2929.01 |
| | 10/30AS | PRE | 13 | 13 | 100 | 75.3 | 100 | 232.25 | 132.26 | 407.85 |
| | | PI(D7) | 12 | 12 | 100 | 73.5 | 100 | 920.78 | 539.84 | 1570.55 |
| | | PI(D14) | 13 | 13 | 100 | 75.3 | 100 | 1569.68 | 980.44 | 2513.05 |
| | | PI(D30) | 14 | 14 | 100 | 76.8 | 100 | 1251.47 | 800.34 | 1956.89 |
| | | PII(D37) | 13 | 13 | 100 | 75.3 | 100 | 1508.59 | 1021.42 | 2228.12 |
| | | PII(D44) | 13 | 13 | 100 | 75.3 | 100 | 1779.93 | 1287.31 | 2461.06 |
| | | PII(D60) | 13 | 13 | 100 | 75.3 | 100 | 1936.73 | 1356.02 | 2766.13 |
| | SALINE | PRE | 30 | 28 | 93.3 | 77.9 | 99.2 | 284.13 | 181.05 | 445.91 |
| | | PI(D7) | 27 | 26 | 96.3 | 81.0 | 99.9 | 306.37 | 186.96 | 502.02 |
| | | PI(D14) | 28 | 27 | 96.4 | 81.7 | 99.9 | 308.14 | 193.80 | 489.93 |
| | | PI(D30) | 30 | 29 | 96.7 | 82.8 | 99.9 | 285.96 | 187.27 | 436.64 |
| | | PII(D37) | 24 | 23 | 95.8 | 78.9 | 99.9 | 268.62 | 160.19 | 450.46 |
| | | PII(D44) | 23 | 22 | 95.7 | 78.1 | 99.9 | 281.86 | 173.60 | 457.65 |
| | | PII(D60) | 24 | 22 | 91.7 | 73.0 | 99.0 | 260.11 | 153.51 | 440.75 |

5/10 = 5 μg CPS5-TT, 5 μg CPS8-TT, 10 μg ClfA, 10 μg α-toxoid
5/10AS = 5 μg CPS5-TT, 5 μg CPS8-TT, 10 μg ClfA, 10 μg α-toxoid adjuvanted with AS03B
10/30 = 10 μg CPS5-TT, 10 μg CPS8-TT, 30 μg ClfA, 30 μg α-toxoid
10/30AS = 10 μg CPS5-TT, 10 μg CPS8-TT, 30 μg ClfA, 30 μg α-toxoid adjuvanted with AS03B
SALINE = pooling of SALINE1 and SALINE2
GMC = geometric mean antibody concentration calculated on all subjects
N = number of subjects with available results
n/% = number/percentage of subjects with concentration within the specified range
95% CI = 95% confidence interval;
LL = Lower Limit,
UL = Upper Limit
PRE = pre dose 1
PI(D7) = 7 days post dose 1
PI(D14) = 14 days post dose 1
PI(D30) = 30 days post dose 1 (blood sample taken at Visits 5 or 6)
PII(D37) = 7 days post dose 2
PII(D44) = 14 days post dose 2
PII(D60) = 30 days post dose 2

TABLE 4
Seropositivity rates and GMCs for Staph aureus ClfA
Ab.IgG antibodies (ATP cohort for immunogenicity)

| | | | | ≧6 ELU/ml | | | | GMC | | |
|---|---|---|---|---|---|---|---|---|---|---|
| | | | | | | 95% CI | | | 95% CI | |
| Antibody | Group | Timing | N | n | % | LL | UL | value | LL | UL |
| Staph aureus.ClfA | 5/10 | PRE | 15 | 15 | 100 | 78.2 | 100 | 58.10 | 31.62 | 106.74 |
| Ab.IgG | | PI(D7) | 15 | 15 | 100 | 78.2 | 100 | 364.64 | 150.30 | 884.67 |
| | | PI(D14) | 14 | 14 | 100 | 76.8 | 100 | 2830.51 | 958.28 | 8360.54 |
| | | PI(D30) | 15 | 15 | 100 | 78.2 | 100 | 3785.71 | 1599.23 | 8961.54 |
| | | PII(D37) | 10 | 10 | 100 | 69.2 | 100 | 4495.84 | 2297.39 | 8798.06 |
| | | PII(D44) | 10 | 10 | 100 | 69.2 | 100 | 5472.85 | 3165.82 | 9461.09 |
| | | PII(D60) | 10 | 10 | 100 | 69.2 | 100 | 4889.94 | 2758.53 | 8668.20 |
| | 5/10AS | PRE | 15 | 15 | 100 | 78.2 | 100 | 128.80 | 81.19 | 204.34 |
| | | PI(D7) | 15 | 15 | 100 | 78.2 | 100 | 1271.87 | 629.74 | 2568.79 |
| | | PI(D14) | 15 | 15 | 100 | 78.2 | 100 | 5967.39 | 3036.36 | 11727.76 |
| | | PI(D30) | 15 | 15 | 100 | 78.2 | 100 | 6580.65 | 3474.92 | 12462.12 |
| | | PII(D37) | 12 | 12 | 100 | 73.5 | 100 | 9654.46 | 5153.40 | 18086.81 |
| | | PII(D44) | 12 | 12 | 100 | 73.5 | 100 | 9852.33 | 5477.46 | 17721.43 |
| | | PII(D60) | 12 | 12 | 100 | 73.5 | 100 | 9875.62 | 5738.09 | 16996.56 |
| | 10/30 | PRE | 14 | 14 | 100 | 76.8 | 100 | 101.38 | 70.70 | 145.39 |
| | | PI(D7) | 14 | 14 | 100 | 76.8 | 100 | 861.08 | 471.92 | 1571.15 |
| | | PI(D14) | 14 | 14 | 100 | 76.8 | 100 | 6627.23 | 3291.32 | 13344.28 |
| | | PI(D30) | 14 | 14 | 100 | 76.8 | 100 | 8068.07 | 4029.42 | 16154.63 |
| | | PII(D37) | 12 | 12 | 100 | 73.5 | 100 | 8465.30 | 4124.58 | 17374.21 |
| | | PII(D44) | 12 | 12 | 100 | 73.5 | 100 | 9130.37 | 4769.02 | 17480.23 |
| | | PII(D60) | 12 | 12 | 100 | 73.5 | 100 | 9840.83 | 5320.61 | 18201.28 |
| | 10/30AS | PRE | 14 | 14 | 100 | 76.8 | 100 | 86.57 | 56.65 | 132.29 |
| | | PI(D7) | 14 | 14 | 100 | 76.8 | 100 | 1097.71 | 550.91 | 2187.24 |
| | | PI(D14) | 14 | 14 | 100 | 76.8 | 100 | 6472.06 | 3731.51 | 11225.35 |
| | | PI(D30) | 14 | 14 | 100 | 76.8 | 100 | 6376.38 | 3505.45 | 11598.55 |
| | | PII(D37) | 13 | 13 | 100 | 75.3 | 100 | 6673.11 | 3836.01 | 11608.50 |
| | | PII(D44) | 13 | 13 | 100 | 75.3 | 100 | 7724.57 | 4739.23 | 12590.44 |
| | | PII(D60) | 13 | 13 | 100 | 75.3 | 100 | 8067.05 | 4906.74 | 13262.83 |
| | SALINE | PRE | 30 | 28 | 93.3 | 77.9 | 99.2 | 80.71 | 46.62 | 139.75 |
| | | PI(D7) | 30 | 28 | 93.3 | 77.9 | 99.2 | 83.79 | 48.36 | 145.18 |
| | | PI(D14) | 30 | 29 | 96.7 | 82.8 | 99.9 | 87.81 | 51.46 | 149.82 |
| | | PI(D30) | 30 | 28 | 93.3 | 77.9 | 99.2 | 91.86 | 52.48 | 160.77 |
| | | PII(D37) | 24 | 22 | 91.7 | 73.0 | 99.0 | 78.61 | 40.78 | 151.52 |
| | | PII(D44) | 23 | 21 | 91.3 | 72.0 | 98.9 | 83.41 | 43.67 | 159.32 |
| | | PII(D60) | 24 | 22 | 91.7 | 73.0 | 99.0 | 81.29 | 42.24 | 156.47 |

5/10 = 5 μg CPS5-TT, 5 μg CPS8-TT, 10 μg ClfA, 10 μg α-toxoid
5/10AS = 5 μg CPS5-TT, 5 μg CPS8-TT, 10 μg ClfA, 10 μg α-toxoid adjuvanted with AS03B
10/30 = 10 μg CPS5-TT, 10 μg CPS8-TT, 30 μg ClfA, 30 μg α-toxoid
10/30AS = 10 μg CPS5-TT, 10 μg CPS8-TT, 30 μg ClfA, 30 μg α-toxoid adjuvanted with AS03B
SALINE = pooling of SALINE1 and SALINE2
GMC = geometric mean antibody concentration calculated on all subjects
N = number of subjects with available results
n/% = number/percentage of subjects with concentration within the specified range
95% CI = 95% confidence interval;
LL = Lower Limit,
UL = Upper Limit
PRE = pre dose 1
PI(D7) = 7 days post dose 1
PI(D14) = 14 days post dose 1
PI(D30) = 30 days post dose 1 (blood sample taken at Visits 5 or 6)
PII(D37)= 7 days post dose 2
PII(D44) = 14 days post dose 2
PII(D60) = 30 days post dose 2

TABLE 5
Seropositivity rates and GMCs for C tetani.Tox
Ab.IgG antibodies (ATP cohort for immunogenicity)

| | | | | ≧0.1 IU/ml | | | | GMC | | |
|---|---|---|---|---|---|---|---|---|---|---|
| | | | | | | 95% CI | | | 95% CI | |
| Antibody | Group | Timing | N | n | % | LL | UL | value | LL | UL |
| C tetani.Tox | 5/10 | PRE | 15 | 13 | 86.7 | 59.5 | 98.3 | 1.071 | 0.366 | 3.139 |
| Ab.IgG | | PI(D7) | 15 | 15 | 100 | 78.2 | 100 | 5.125 | 2.687 | 9.777 |
| | | PI(D14) | 14 | 14 | 100 | 76.8 | 100 | 11.070 | 7.188 | 17.047 |
| | | PI(D30) | 15 | 15 | 100 | 78.2 | 100 | 8.324 | 5.200 | 13.325 |
| | | PII(D37) | 10 | 10 | 100 | 69.2 | 100 | 7.516 | 3.585 | 15.756 |
| | | PII(D44) | 10 | 10 | 100 | 69.2 | 100 | 6.909 | 3.469 | 13.757 |
| | | PII(D60) | 10 | 10 | 100 | 69.2 | 100 | 5.582 | 2.473 | 12.601 |
| | 5/10AS | PRE | 15 | 14 | 93.3 | 68.1 | 99.8 | 2.010 | 0.879 | 4.600 |
| | | PI(D7) | 15 | 15 | 100 | 78.2 | 100 | 7.096 | 4.799 | 10.494 |
| | | PI(D14) | 15 | 15 | 100 | 78.2 | 100 | 10.545 | 7.732 | 14.382 |
| | | PI(D30) | 15 | 15 | 100 | 78.2 | 100 | 9.249 | 6.845 | 12.497 |
| | | PII(D37) | 12 | 12 | 100 | 73.5 | 100 | 8.530 | 6.265 | 11.615 |
| | | PII(D44) | 12 | 12 | 100 | 73.5 | 100 | 8.906 | 5.604 | 14.154 |
| | | PII(D60) | 12 | 12 | 100 | 73.5 | 100 | 8.600 | 5.470 | 13.521 |
| | 10/30 | PRE | 14 | 13 | 92.9 | 66.1 | 99.8 | 3.264 | 1.225 | 8.698 |
| | | PI(D7) | 14 | 14 | 100 | 76.8 | 100 | 16.200 | 10.728 | 24.463 |
| | | PI(D14) | 14 | 14 | 100 | 76.8 | 100 | 22.716 | 14.191 | 36.364 |
| | | PI(D30) | 14 | 14 | 100 | 76.8 | 100 | 16.495 | 10.461 | 26.010 |
| | | PII(D37) | 12 | 12 | 100 | 73.5 | 100 | 17.044 | 10.457 | 27.778 |
| | | PII(D44) | 12 | 12 | 100 | 73.5 | 100 | 16.647 | 9.980 | 27.767 |
| | | PII(D60) | 12 | 12 | 100 | 73.5 | 100 | 14.762 | 9.029 | 24.134 |
| | 10/30AS | PRE | 14 | 14 | 100 | 76.8 | 100 | 3.307 | 2.344 | 4.664 |
| | | PI(D7) | 14 | 14 | 100 | 76.8 | 100 | 14.276 | 9.854 | 20.683 |
| | | PI(D14) | 14 | 14 | 100 | 76.8 | 100 | 16.527 | 12.036 | 22.693 |
| | | PI(D30) | 14 | 12 | 85.7 | 57.2 | 98.2 | 5.479 | 1.671 | 17.963 |
| | | PII(D37) | 13 | 13 | 100 | 75.3 | 100 | 13.042 | 9.511 | 17.883 |
| | | PII(D44) | 13 | 13 | 100 | 75.3 | 100 | 12.104 | 8.706 | 16.828 |
| | | PII(D60) | 13 | 13 | 100 | 75.3 | 100 | 11.461 | 8.396 | 15.647 |
| | SALINE | PRE | 30 | 29 | 96.7 | 82.8 | 99.9 | 1.779 | 1.171 | 2.704 |
| | | PI(D7) | 30 | 29 | 96.7 | 82.8 | 99.9 | 1.831 | 1.198 | 2.797 |
| | | PI(D14) | 30 | 29 | 96.7 | 82.8 | 99.9 | 1.968 | 1.295 | 2.989 |
| | | PI(D30) | 30 | 28 | 93.3 | 77.9 | 99.2 | 1.705 | 1.055 | 2.757 |
| | | PII(D37) | 24 | 23 | 95.8 | 78.9 | 99.9 | 1.932 | 1.159 | 3.220 |
| | | PII(D44) | 23 | 22 | 95.7 | 78.1 | 99.9 | 1.929 | 1.133 | 3.286 |
| | | PII(D60) | 24 | 23 | 95.8 | 78.9 | 99.9 | 2.001 | 1.185 | 3.378 |

5/10 = 5 μg CPS5-TT, 5 μg CPS8-TT, 10 μg ClfA, 10 μg α-toxoid
5/10AS = 5 μg CPS5-TT, 5 μg CPS8-TT, 10 μg ClfA, 10 μg α-toxoid adjuvanted with AS03B
10/30 = 10 μg CPS5-TT, 10 μg CPS8-TT, 30 μg ClfA, 30 μg α-toxoid
10/30AS = 10 μg CPS5-TT, 10 μg CPS8-TT, 30 μg ClfA, 30 μg α-toxoid adjuvanted with AS03B
SALINE = pooling of SALINE1 and SALINE2
GMC = geometric mean antibody concentration calculated on all subjects
N = number of subjects with available results
n/% = number/percentage of subjects with concentration within the specified range
95% CI = 95% confidence interval;
LL = Lower Limit,
UL = Upper Limit
PRE = pre dose 1
PI(D7) = 7 days post dose 1
PI(D14) = 14 days post dose 1
PI(D30) = 30 days post dose 1 (blood sample taken at Visits 5 or 6)
PII(D37) = 7 days post dose 2
PII(D44) = 14 days post dose 2
PII(D60) = 30 days post dose 2

### Example 4—Stability of Vaccine Components

Immunogenic compositions comprising ClfA, alpha toxoid, capsular polysaccharide type 5 and capsular polysaccharide type 8 were subjected to stability tests when formulated at different pH conditions and different salt conditions. The components were tested under the following conditions:

10 mM phosphate buffer pH 5.5, 150 mM NaCl
10 mM phosphate buffer pH 7.5, 150 mM NaCl
10 mM phosphate buffer pH6.5, 50 mM NaCl
10 mM phosphate buffer pH 6.5, 450 mM NaCl
10 mM phosphate buffer pH 6.5, 150 mM NaCl
10 mM phosphate buffer p 6.5, 150 mM NaCl, 0.05% Triton
10 mM phosphate buffer pH 6.5, 150 mM NaCl, 0.01% Tween

The samples containing either S. aureus capsular polysaccharide and alpha toxoid or S. aureus capsular polysaccharide type 8 and ClfA were incubated at 4 degrees C. for seven days. The components were separated by SDS-PAGE and the level of impurities was measured by comparing the intensities of the expected band and degradation products.

The following levels of purity were found:

TABLE 6

| Type 5CPS- | | | | | |
|---|---|---|---|---|---|
| alpha tox | | | | | |
| condition | pH 5.5, | pH 7.5, | pH 6.5, | pH 6.5, | pH 6.5, |
| | 150 mM | 150 mM | 50 mM | 450 mM | 150 mM |
| Purity | 95.6% | 94.8% | 95.2% | 94.5% | 95.0% |
| Type 8CPS- | | | | | |
| ClfA | | | | | |
| Condition | pH 5.5, | pH 7.5, | pH 6.5, | pH 6.5, | pH 6.5, |
| | 150 mM | 150 mM | 50 mM | 450 mM | 150 mM |
| Purity | 95.6% | 95.2% | 95.1% | 94.8% | 95.1% |

A further experiment was carried out using the 10 mM phosphate buffer pH 6.5, 50 mM NaCl solution. Samples containing S. aureus capsular polysaccharide 5—alpha toxoid or S. aureus capsular polysaccharide 8—ClfA were stored for 7 days at 4 degrees C. or 37 degrees C. The samples were then run on an SDS-PAGE and the level of impurities was measured by comparing the intensities of the expected band and degradation products. The following results were found:

TABLE 7

| Type 5CPS-alpha tox | | |
|---|---|---|
| Condition | pH 6.5, 50 mM NaCl | pH 6.5, 50 mM NaCl |
| | 4 degrees C. | 37 degrees C. |
| Purity | 89.2% | 89.0% |
| Type 8CPS-ClfA | | |
| Condition | pH 6.5, 50 mM NaCl | pH 6.5, 50 mM NaCl |
| | 4 degrees C. | 37 degrees C. |
| Purity | 95.7% | 96.4% |

No evidence of instability was seen for the staphylococcal components tested when formulated at pH 5.5-pH 7.5 in the presence of 50 mM-450 mM NaCl.

1. A method of treating or preventing a Staphylococcus aureus infection in a human patient, comprising administering to the patient a single dose of an immunogenic composition comprising; (i) a Staphylococcus aureus ClfA protein or immunogenic fragment thereof at a dose of 5-50, 10-30, 5-15 or 20-40 μg, and (ii) a pharmaceutically acceptable excipient; wherein the pH of the immunogenic composition is pH 5.0-8.0.

2. The method of claim 1 wherein the ClfA protein or immunogenic fragment thereof is at least 90% identical to the polypeptide sequence of any one of SEQ ID NO:3-12 or 15-18 along the full length thereof.

3. The method of claim 1 wherein the ClfA protein or immunogenic fragment thereof is a fragment of ClfA comprising a N2 domain.

4. The method of claim 1 wherein the ClfA protein or immunogenic fragment thereof is a fragment of ClfA comprising a N3 domain.

5. The method of claim 1 wherein the ClfA protein or immunogenic fragment thereof is a fragment of ClfA comprising a N1 domain.

6. The method of claim 1 wherein the ClfA protein or immunogenic fragment thereof contains an amino acid substitution which reduces the ability of ClfA to bind to fibrinogen.

7. The method of claim 6 wherein the ClfA protein or immunogenic fragment thereof contains an amino acid substitution at at least one of amino acids Ala254, Tyr256, Pro336, Tyr338, Ile387, Lys389, Tyr474, Glu526 or Val527.

8. The method of claim 1 wherein the ClfA protein or immunogenic fragment thereof is present in the immunogenic composition at a dose of 20-40 μg.

9. The method of claim 1 wherein the immunogenic composition comprises an alpha toxoid protein.

10. The method of claim 9 wherein the alpha toxoid protein has an amino acid sequence at least 90% identical to SEQ ID NO:1, 2, 13 or 14.

11. The method of claim 9 wherein the alpha toxoid protein is present in the immunogenic composition at a dose of 5-50, 10-30, 5-15 or 20-40 μg.

12. The method of claim 1 wherein the single dose of the immunogenic composition is administered 5-60, 6-40, 7-30 or 7-15 days before a planned hospital procedure.

13. A method of treating or preventing a Staphylococcus aureus infection in a human patient, comprising administering to the patient a single dose of an immunogenic composition comprising; (i) a Staphylococcus aureus alpha toxoid protein or immunogenic fragment thereof at a dose of 5-50, 10-30, 5-15 or 20-40 μg, and (ii) a pharmaceutically acceptable excipient; wherein the pH of the immunogenic composition is pH 5.0-pH 8.0.

14. The method of claim 13, wherein the alpha toxoid protein or immunogenic fragment thereof has an amino acid sequence at least 90% identical to SEQ ID NO:1, 2, 13 or 14.

15. An immunogenic composition comprising; (i) a S. aureus Type 5 capsular saccharide conjugated to a carrier protein, (ii) a S. aureus Type 8 capsular saccharide conjugated to a carrier protein, (iii) a ClfA protein or immunogenic fragment thereof, (iv) an alpha toxoid, and (v) a pharmaceutically acceptable excipient; wherein the pH of the immunogenic composition is pH 5.0-pH 8.0.

16. A process for making the immunogenic composition of claim 15 comprising the steps of a) conjugating a S. aureus Type 5 capsular saccharide to a carrier protein to form a S. aureus Type 5 capsular saccharide conjugate, b) conjugating a S. aureus Type 8 capsular saccharide conjugated to a carrier protein to form a S. aureus Type 8 capsular saccharide conjugate, and c) combining the S. aureus Type 5 capsular saccharide conjugate, the S. aureus Type 8 capsular saccharide conjugate, a ClfA protein or immunogenic fragment thereof and an alpha toxoid to form the immunogenic composition.

17. The method of claim 13, wherein the single dose of the immunogenic composition is administered 5-60, 6-40, 7-30 or 7-15 days before a planned hospital procedure.

18. The method of claim 9, wherein the single dose of the immunogenic composition further comprises a S. aureus Type 5 capsular saccharide conjugated to a carrier protein and a S. aureus Type 8 capsular saccharide conjugated to a carrier protein.


SEQUENCE LISTING (18 sequences)

SEQ ID NO: 1 - protein, 319 aa, Staphylococcus aureus
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 2 - protein, 294 aa, Staphylococcus aureus
MADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 3 - protein, 927 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSDNDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSDSDSDSDSDSDSDSDSDSDSDSDSDSDSDSESDSDSDSDSDSDSDSDSDSDSDSASDSDSGSDSDSSSDSDSESDSNSDSESVSNNNVVPPNSPKNGTNASNKNEAKDSKEPLPDTGSEDEANTSLIWGLLASIGSLLLFRRKKENKDKK

SEQ ID NO: 4 - protein, 521 aa, Staphylococcus aureus
MSENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 5 - protein, 559 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 6 - protein, 343 aa, Staphylococcus aureus
SLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 7 - protein, 331 aa, Staphylococcus aureus
GTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 8 - protein, 927 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEDSDSDPGSDSGSDSNSDSGSDSGSDSTSDSGSDSASDSDSASDSDSASDSDSASDSDSASDSDSDNDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSDSASDSDSDSDSDSDSDSDSDSDSDSDSDSDSDSDSDSDSDSESDSDSDSDSDSDSDSDSDSDSDSASDSDSGSDSDSSSDSDSESDSNSDSESVSNNNVVPPNSPKNGTNASNKNEAKDSKEPLPDTGSEDEANTSLIWGLLASIGSLLLFRRKKENKDKK

SEQ ID NO: 9 - protein, 521 aa, Staphylococcus aureus
MSENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 10 - protein, 559 aa, Staphylococcus aureus
MNMKKKEKHAIRKKSIGVASVLVGTLIGFGLLSSKEADASENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 11 - protein, 343 aa, Staphylococcus aureus
SLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 12 - protein, 331 aa, Staphylococcus aureus
GTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 13 - protein, 319 aa, Staphylococcus aureu
MKTRIVSSVTTTLLLGSILMNPVANAADSDINIKTGTTDIGSNTTVKTGDLVTYDKENGMRKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 14 - protein, 294 aa, Staphylococcus aureus
MADSDINIKTGTTDIGSNTTVKTGDLVTYDKENGMRKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 15 - protein, 345 aa, Staphylococcus aureus
MASLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 16 - protein, 333 aa, Staphylococcus aureus
MAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 17 - protein, 345 aa, Staphylococcus aureus
MASLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 18 - protein, 333 aa, Staphylococcus aureus
MAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE